(19) United States
(12) Patent Application Publication
(10) Pub. No.: US 2017/0253881 A1
(43) Pub. Date: Sep. 7, 2017

(54) COMPOSITIONS AND METHODS OF SELECTIVELY INHIBITING IRP1 AND TREATING INFLAMMATION

(71) Applicant: Technion Research & Development Foundation Limited, Haifa (IL)

(72) Inventors: Esther MEYRON-HOLTZ, Haifa (IL); Shirly MOSHE-BELIZOWSKY, Haifa (IL)

(73) Assignee: Technion Research & Development Foundation Limited, Haifa (IL)

(21) Appl. No.: 15/314,563

(22) PCT Filed: Jun. 2, 2015
(86) PCT No.: PCT/IL2015/050565

§ 371 Date: Nov. 29, 2016

Related U.S. Application Data
(60) Provisional application No. 62/006,326, filed on Jun. 2, 2014.

(51) Int. Cl. C12N 15/113; G06F 19/16; C12Q 1/527; A61K 38/02; C12Q 1/68

(52) U.S. Cl. CPC C12N 15/1137; A61K 38/02; C12Q 1/6883; C12Y 402/01003; C12Q 1/527; G06F 19/16; C12N 2320/11; C12N 2310/14; C12N 2310/531; G01N 2333/988; G01N 2500/10; G01N 2500/04; G01N 2800/065; G01N 2800/102; G01N 2800/7095

(57) ABSTRACT

Compositions and methods of treating an inflammatory disease in a subject are provided. Accordingly there is provided a method comprising administering to the subject a therapeutically effective amount of an agent which selectively inhibits activity and/or expression of iron regulatory protein (IRP) 1 and not IRP2, thereby treating the inflammatory disease in the subject. Also provided is a pharmaceutical composition comprising, as an active ingredient, an agent which selectively inhibits activity and/or expression of IRP1 and not IRP2, and a pharmaceutically acceptable carrier or excipient. Also provided are methods of identifying an agent that selectively modulates an activity of an IRP member of an IRP family of polypeptides and not of an additional IRP member of said IRP family of polypeptides.

### FIELD AND BACKGROUND OF THE INVENTION

The present invention, in some embodiments thereof, relates to compositions and methods of selectively inhibiting iron regulatory protein (IRP) 1 and treating inflammation.

Chronic and acute inflammatory diseases such as inflammatory bowel disease (IBD), rheumatoid arthritis (RA) and chronic kidney disease represent a substantial health concern. Treatment options for patients suffering from inflammatory disease include conventional therapies such as antibiotics, corticosteroids, immunosuppressants and biologics such as anti-TNFα compounds, and in severe cases hospitalization and surgery.

Iron is an essential nutrient involved in many central processes in the body, such as oxygen transport, electron transport in the respiratory chain and DNA synthesis. As iron readily exchanges electrons it is widely used for a number of enzymatic functions requiring the transfer of electrons via oxidation-reduction reactions. However, excess of free iron is toxic to cells due to its ability to form reactive oxygen species through the Fenton reaction. The dual role of iron has led to the evolution of a complex network of transporters, storage molecules, and regulators that coordinately govern iron absorption, recycling, and mobilization as well as maintain iron homeostasis at both cell and systemic levels. [Recalcati et al. Antioxidants & Redox signaling (2010) 13: 1593-1616]

Iron homeostasis, and specifically, the regulation of iron control elements has been indicated to be involved in inflammatory diseases [e.g. Yamaji et al. Blood (2004) 104: 2178-2180]. Thus, for example, RA patients exhibit higher concentration of free iron in the synovial fluid and activity of iron control elements in cells isolated from their synovial fluid correlates with serum c-reactive protein (CRP), which is a marker of inflammation [Yazar M, et al. (2005) Biological trace element research 106(2):123-132; and Guillen C, et al. Ann Rheum Dis (1998) 57:309-314].

Iron homeostasis at the cellular level is mainly maintained by iron regulatory proteins IRP1 and IRP2. Both IRPs are ubiquitously expressed cytosolic proteins that act by binding to cis-regulatory mRNA motifs called iron responsive elements (IREs), identified in the untranslated regions (UTRs) of mRNAs of several proteins involved in iron uptake, utilization, storage, and export, such as DMT1, transferrin and ferritin. In general, IRPs bind to the respective target mRNA under low cytosolic iron concentrations, albeit it has been shown that IRP1 and IRP2 respond to changing iron levels with different mechanisms. [Meyron-Holtz et al. EMBO J. (2004) 23:386-95] The type of regulation depends on the location of the IRE in the target mRNA. Binding of either IRPs to the IRE in the 5′ UTR prevents translation, whereas binding to the IRE in the 3′ UTR increases mRNA stability. The cumulative effect of the binding of the IRPs to the IREs leads eventually to increased iron uptake and both its intracellular and whole-body availability, while decreased IRE-binding activity leads ultimately to decreased intracellular iron levels. [Recalcati et al. Antioxidants & Redox signaling (2010) 13:1593-1616].

IRP1 is a bifunctional protein: when cytosolic iron levels are high IRP1 contains a [4Fe-4S] cluster, and has cytosolic aconitase activity but cannot bind to IREs; while under low iron conditions, the [4Fe-4S] cluster is disassembled, and IRP1 loses its aconitase activity and acquires IRE-binding capacity thereby regulating an increase in translation of e.g. transferrin receptor and DMT1 and inhibiting translation of e.g. ferritin leading eventually to increased iron uptake and availability. [Koskenkorva-Frank et al. Free Radical Biology and Medicine (2013) 65:1174-1194].

IRP-2 does not assemble an Fe—S cluster and spontaneously binds IREs. When iron levels are high, F-box and leucine-rich repeat protein 5 (FBXL5) binds to its target motifs on IRP2 and induces its proteasomal degradation. Under conditions of low iron levels, FBXL5 itself is targeted for ubiquitination and degraded, which stabilizes IRP2 and allows its binding to IREs [Koskenkorva-Frank et al. Free Radical Biology and Medicine (2013) 65:1174-1194].

The binding activity of IRPs is also controlled by several factors other than iron, for instance, tissue oxygen level, oxidative stress, and nitrosative stress. Thus, both IRPs are sensitive to cellular oxygen concentrations, but in an inverse fashion: IRP2 mainly reacts to high oxygen and reactive oxygen and nitrogen species with increased degradation while IRP1 needs a high oxygen concentration to function as an RNA binding protein, therefore IRP2 is the dominant iron regulator in normal physiological conditions [Meyron-Holtz et al. Science (2004) 306:2087-90]. It has also been shown that IRP1 and IRP2 can bind to distinct sets of mRNAs [Henderson et al. J. Biol. Chem. (1996) 271: 4900-4908].

Mouse embryos lacking both IRPs die early during embryonic life, thus indicating that the IRE-IRP regulatory system is essential for development. By contrast, animals lacking either protein are viable and fertile. IRP1−/− mice do not exhibit serious pathologies under normal physiologic conditions. These mice develop a transient increase in hematocrit, misregulate transferrin receptor 1 and ferritin expression in the kidney and brown fat, which are IRP1-enriched tissues and only show more serious pathologies when stressed by very low iron diets. [Ghosh et al. Cell Metab. 2013 Feb. 5; 17(2):271-81; Anderson et al. Cell Metab. 2013 Feb. 5; 17(2):282-90 and Wilkinson et al. Blood. 2013 Aug. 29; 122(9): 1658-68].

### ADDITIONAL RELATED ART

Xavier et al. Trends Biotech., (2000) 18: 349-356;
Ecker and Giffey Drug Discov Today. (1999) 4(9):420-429;
Tibodeau et al. PNAS (2006) 103 (2): 253-257;
US Patent Publication No. 20110281744;
Venti et al. Ann. N.Y. Acad. Sci. (2004) 1035: 34-48;
Bandyopadhyay et al. PLoS One. (2013) 8(7):e65978;
Zimmer et al. Cancer Res 2010; 70:3071-3079;
US Publication No. 20120070369;
Mastrogiannaki et al. Blood (2013) 122(6): 885-892;
Sourbier et al. Oncotarget. (2012) 3(11):1472-82;
Wang et al. Cancer Res. (2014) 74(2):497-507;
Anderson et al. Cell Metabolism (2013) 17, 282-290;
Chen et al. Carcinogenesis (2007) 28: 785-791;
Pantopoulos et al. Biochemistry (2012) 51(29): 5705-5724;
Stys' et al. J. Biol. Chem. (2011) 286:22846-22854;
Meyron-Holtz et al. Oral presentation at the International Biolron Society
Meeting, Jun. 7-11, 2009, Porto, Portugal;
Moshe Belizowsky and Meyron-Holtz, Oral presentation at the European Iron Club, Sep. 8, 2011, Louvain-la-Neuve, Belgium;
Moshe Belizowsky et al. Poster at the Biolron, April 2013, Technion-Israel Institute of Technology;
Savion et al. Oral presentation at ISOFRR, 28. Dec. 2008, Israel;
Reifen and Meyron-Holtz Poster at the First International Conference On Metal Chelation in Biology & Medicine. December 2009, Bath, United Kingdom; and
Reifen et al. Poster at the Falk Symposium 168 IBD in Different Age Groups, March 2009, Madrid, Spain.

### SUMMARY OF THE INVENTION

According to an aspect of some embodiments of the present invention there is provided a method of treating an inflammatory disease in a subject in need thereof, the method comprising administering to the subject a therapeutically effective amount of an agent which selectively inhibits activity and/or expression of iron regulatory protein (IRP) 1 and not IRP2, thereby treating the inflammatory disease in the subject.

According to an aspect of some embodiments of the present invention there is provided an agent, which selectively inhibits activity and/or expression of IRP1 and not IRP2, for use in the treatment of an inflammatory disease in a subject.

According to an aspect of some embodiments of the present invention there is provided a pharmaceutical composition comprising, as an active ingredient, an agent which selectively inhibits activity and/or expression of IRP1 and not IRP2, and a pharmaceutically acceptable carrier or excipient.

According to some embodiments of the invention the activity is binding to an iron responsive element (IRE).

According to some embodiments of the invention the inflammatory disease is selected from the group consisting of an autoimmune disease, an infectious disease, cancer and a neurodegenerative disease.

According to some embodiments of the invention the inflammatory disease comprises inflammatory bowel disease (IBD).

According to some embodiments of the invention the inflammatory disease comprises rheumatoid arthritis (RA).

According to some embodiments of the invention the inflammatory disease is an oxidative stress disease.

According to an aspect of some embodiments of the present invention there is provided a method of identifying an agent that selectively modulates an activity of an IRP member of an IRP family of polypeptides and not of an additional IRP member of the IRP family of polypeptides, the method comprising:
(a) determining an effect of a test agent on binding of the IRP member to at least one nucleic acid sequence containing an IRE and/or on expression of a reporter gene comprising the at least one nucleic acid sequence containing the IRE; and
(b) determining an effect of the test agent on binding of the additional IRP member to at least one nucleic acid sequence containing the IRE and/or on expression of the reporter gene comprising the at least one nucleic acid sequence containing the IRE; wherein:
(i) an alteration in binding of the IRP member to the at least one nucleic acid sequence containing the IRE and/or expression of the reporter gene comprising the at least one nucleic acid sequence containing the IRE nucleic acid sequence as compared to same in an absence of the test agent; and
(ii) no alteration in binding of the additional IRP member to the at least one nucleic acid sequence containing the IRE and/or expression of the reporter gene comprising the at least one nucleic acid sequence containing the IRE as compared to same in an absence of the test agent,
are indicative of an agent that selectively modulates activity of an IRP member of an IRP family of polypeptides and not of an additional IRP member of the IRP family of polypeptides.

According to an aspect of some embodiments of the present invention there is provided a method of identifying an agent that selectively inhibits an activity of an IRP1, the method comprising:
(a) in-silico selecting a test agent that inhibits binding of an RNA binding form of IRP1 to an IRE but does not inhibit of IRP2 to an IRE; or
(b) in-silico selecting a test agent stabilizing a 4Fe-4S cluster form of IRP1.

According to some embodiments of the invention the method further comprises providing said test agent and testing an anti-inflammatory activity of same.

According to some embodiments of the invention, said testing is effected in-vitro. According to some embodiments of the invention the IRP member is IRP1 and the additional IRP member is IRP2.

According to some embodiments of the invention the IRP member is IRP2 and the additional IRP member is IRP1.

According to some embodiments of the invention modulates the activity is inhibits the activity.

According to some embodiments of the invention modulates the activity is activates the activity.

According to some embodiments of the invention the agent comprises a siRNA or an antisense oligonucleotides.

According to some embodiments of the invention the agent is selected from the group consisting of a peptide and a small molecule.

According to some embodiments of the invention the reporter gene comprising the at least one nucleic acid sequence containing the IRE is a naturally occurring molecule.

According to some embodiments of the invention the reporter gene comprising the at least one nucleic acid sequence containing the IRE is a chimeric molecule.

According to some embodiments of the invention the nucleic acid sequence containing the IRE is of a polynucleotide selected from the group consisting of transferrin receptor IRE, ferritin IRE, and DMT1 IRE.

According to some embodiments of the invention the at least one nucleic acid sequence containing the IRE comprises at least two different nucleic acid sequences containing the IRE.

According to some embodiments of the invention the nucleic acid sequence containing the IRE is positioned upstream of the reporter gene.

According to some embodiments of the invention the nucleic acid sequence containing the IRE is positioned downstream of the reporter gene.

According to some embodiments of the invention one of the at least two different nucleic acid sequences containing the IRE is positioned upstream of the reporter gene and a second of the at least two different nucleic acid sequences containing the IRE is positioned downstream of the reporter gene.

According to some embodiments of the invention the effect on expression is downregulation of the expression.

According to some embodiments of the invention the effect on expression is upregulation of the expression.

According to some embodiments of the invention the effect on expression is downregulation of the expression of the at least one nucleic acid sequence containing the IRE and upregulation of the expression of the at least one nucleic acid sequence containing the IRE.

According to some embodiments of the invention the nucleic acid sequence containing the IRE is attached to a detectable moiety.

According to some embodiments of the invention the determining is effected by an apparatus selected from the group consisting of flow cytometer, fluorescent plate reader and luminescence plate reader.

According to some embodiments of the invention the at least one nucleic acid sequence containing the IRE is comprised in a cell.

According to some embodiments of the invention the cell is not expressing endogenous IRP1 and/or IRP2.

According to some embodiments of the invention the (a) is effected in a cell not expressing endogenous IRP2 and the (b) is effected in a cell not expressing endogenous IRP1.

According to some embodiments of the invention the (a) is effected in a cell not expressing endogenous IRP1 and the (b) is effected in a cell not expressing endogenous IRP2.

According to some embodiments of the invention the method is effected under low iron conditions.

According to some embodiments of the invention the method is effected under high iron conditions.

According to some embodiments of the invention the method is effected under oxidative and/or nitrosative stress conditions.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The patent or application file contains at least one drawing executed in color. Copies of this patent or patent application publication with color drawing(s) will be provided by the Office upon request and payment of the necessary fee.

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-B show that IRP1 is a positive regulator of the intestinal inflammation process in TNF^{ΔARE/+} mice while presence of IRP2 is necessary to avoid inflammation. TNF^{ΔARE/+}, C57Bl6 wild type, IRP1^{-/-}, TNF^{ΔARE/+} on IRP1^{-/-} background TNF^{ΔARE/+} on IRP2^{-/-} background and TNF^{ΔARE/+} on IRP1^{-/-} background heterozygous for IRP2 mice were sacrificed at 12-14 weeks old age and paraffin embedded terminal ileum sections were prepared for histological evaluation. FIG. 1A depicts representative histological images (H&E, magnification ×200) showing that the TNF^{ΔARE/+}, TNF^{ΔARE/+}IRP2−/−, and TNF^{ΔARE/+}IRP1−/−IRP2+/− mice suffer from severe transmural intestinal inflammation while both, IRP1^{-/-} and TNF^{ΔARE/+} mice on IRP1^{-/-} background show no inflammatory phenotype. FIG. 1B is a bar graph demonstrating that the total histological inflammation score of wt, IRP1^{-/-} and TNF^{ΔARE/+} on IRP1^{-/-} background mice is significantly lower compared to TNF^{ΔARE/+} mice. Results are expressed as mean±SD. n=12 for wild type mice, n=9 for TNF^{ΔARE/+} mice, n=5 for IRP1^{-/-} and TNF^{ΔARE/+} mice on IRP1^{-/-} background (the evaluation was performed in a blind fashion). ** P<0.0001.

FIG. 2 depicts representative histological images that demonstrate that iron accumulation is reduced in the intestine of TNF^{ΔARE/+} mice on IRP1^{-/-} background compared to TNF^{ΔARE/+} mice. The assayed mice were subjected to iron overload. TNF^{ΔARE/+}, C57Bl6 wild type, IRP1^{-/-}, and TNF^{ΔARE/+} on IRP1^{-/-} background mice were sacrificed at 12-14 weeks old age. Paraffin embedded terminal ileum sections were prepared and subjected to iron staining. Histological evaluation shows that iron levels are elevated only in the inflamed intestine of TNF^{ΔARE/+} mice and that no iron accumulates in the TNF^{ΔARE/+} mice on IRP1^{-/-} background. The iron accumulation is mainly detected in the immune cells infiltrating the inflamed area and not in the epithelial cells (magnification ×200).

FIGS. 3A-C demonstrate that IRP1 knockout attenuates the effect on iron homeostasis observed during the intestinal inflammation process in TNF^{ΔARE/+} mice. TNF^{ΔARE/+}, C57Bl/6 wild type and TNF^{ΔARE/+} on IRP1^{-/-} background mice were sacrificed at 12-14 weeks old age. Paraffin embedded terminal ileum sections were prepared and subjected to immune-fluorescent staining with an antibody against mouse L-ferritin and DAPI nuclear staining. Representative images (magnification ×200, FIG. 3A) and fluorescence intensity quantification (performed by Imaris software) in the IEC (FIG. 3B) and in the lamina propria immune cells (FIG. 3C) show that ferritin levels are increased in the immune cells, while decreased in the IEC in TNF^{ΔARE/+} mice, while this abnormal regulation is reversed by IRP1 deletion.

FIG. 4 are Western blot photomicrographs showing that expression levels of proteins involved in iron homeostasis are significantly altered in IEC enriched cell-fractions obtained from the terminal ileum of TNF^{ΔARE/+} compared to C57Bl/6 wild type mice. The expression level of β-actin was used as an internal positive control.

FIGS. 5A-B show that IRP1 knockout significantly reduces TNFα transcription and prevents pathological over-expression of TNFα in the terminal ileum of TNF^{ΔARE/+} mice. FIG. 5A is a Western blot photomicrographs showing TNFα protein expression levels in cell extracts obtained from the terminal ileum of C57Bl/6 wild type, TNF^{ΔARE/+}, and TNF^{ΔARE/+} on IRP1^{-/-} background mice. The expression level of β-actin was used as an internal positive control. FIG. 5B is a graph showing TNFα mRNA levels in cell extracts obtained from the terminal ileum of C57Bl/6 wild type, TNF^{ΔARE/+}, IRP1^{-/-} and TNF^{ΔARE/+} on IRP1^{-/-} background mice, as evaluated by qRT-PCR. Bars present mean±SD, n=4 of TNF-α levels normalized to the TNF-α levels in C57Bl/6 wild type, after normalization to β-actin. **p<0.001,***p<0.0001.

FIG. 6 are representative histological images (H&E, magnification ×100) showing that the TNF^{ΔARE/+}IRP1+/− mice suffer from severe synovial inflammation while and TNF^{ΔARE/+} mice on IRP1^{-/-} background show a significantly reduced inflammatory phenotype.

FIG. 7 is a scheme of the proposed mechanism of action of IRP1 knock out on inflammation in TNF^{ΔARE/+} mice.

### DESCRIPTION OF SPECIFIC EMBODIMENTS OF THE INVENTION

The present invention, in some embodiments thereof, relates to compositions and methods of selectively inhibiting iron regulatory protein (IRP) 1 and treating inflammation.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

Iron is an essential nutrient involved in many central processes in the body, however, too much or too little iron is toxic to cells. Therefore, a complex regulatory network controls iron homeostasis at both cell and systemic levels. Iron homeostasis at the cellular level is mainly maintained by iron regulatory proteins IRP1 and IRP2.

Whilst reducing the present invention to practice, the present inventors have uncovered that knock down of IRP1 prevents the development of inflammation as evidenced in animal models for IBD and RA where transmural intestinal inflammation and synovial inflammation in mice carrying a deletion of the TNF AU-rich regulatory element (TNF^{ΔARE/+}) that spontaneously develop inflammatory polyarthritis and inflammatory bowel disease, respectively, were much inhibited. These findings conclusively suggest the use of IRP1 inhibitors in the treatment of an inflammatory disease.

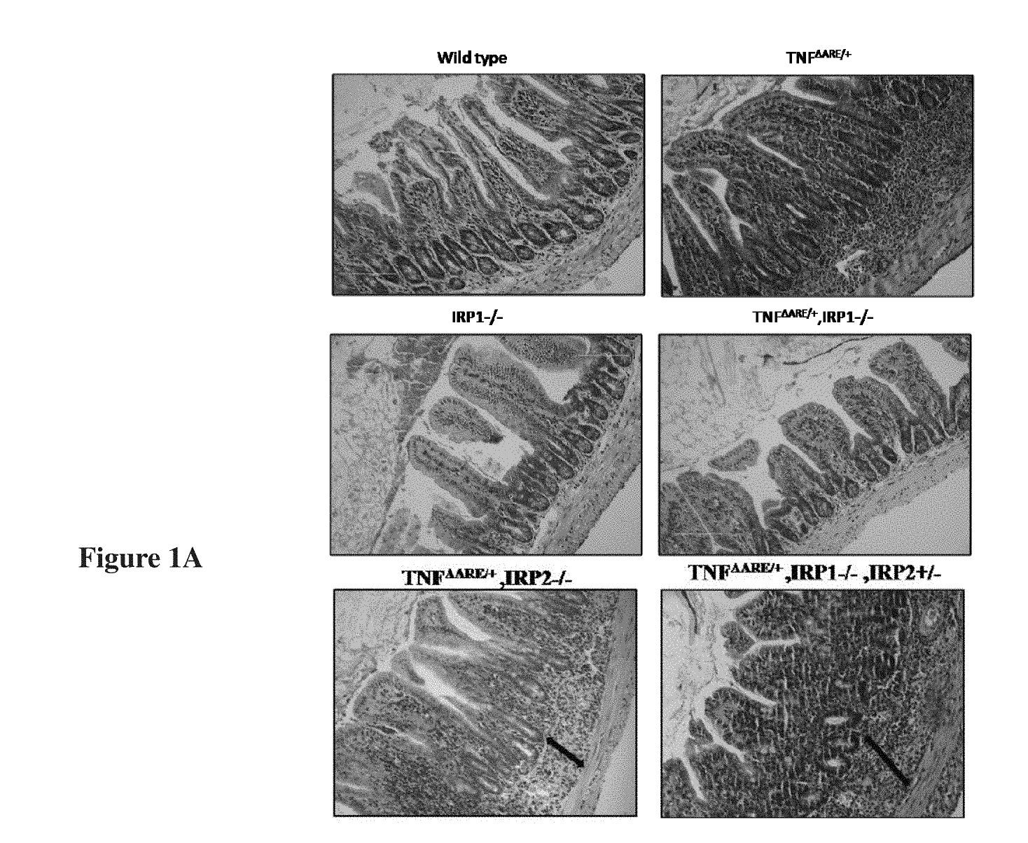

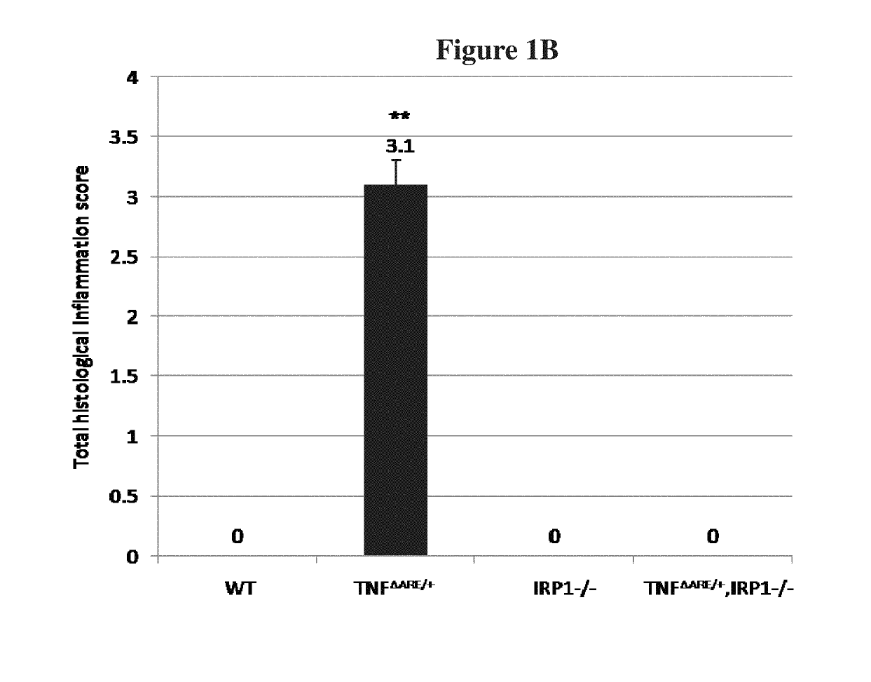

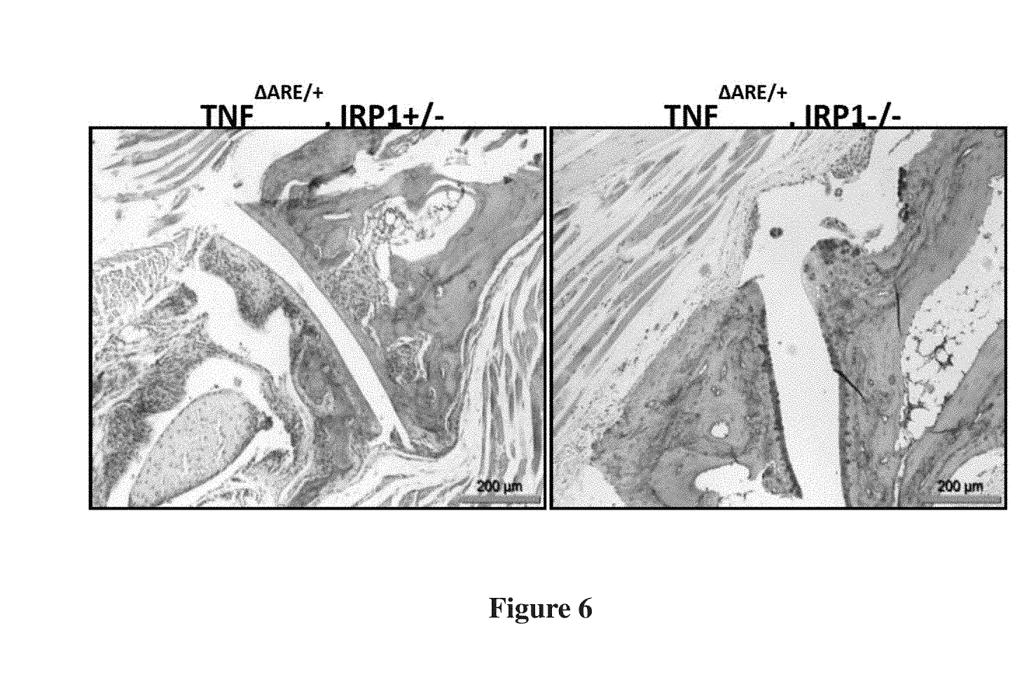

Specifically, the present inventors have uncovered that IRP1 deletion prevents TNFα overexpression, attenuates the alteration in iron homeostasis and completely abolishes the intestinal inflammation in TNF^{ΔARE/+} mice (Example 1, FIGS. 1A-B, 2, 3A-C, 4 and 5A-B). Most importantly, not only that deletion of IRP2 does not prevent the intestinal inflammation in TNF^{ΔARE/+} mice it's deletion completely abolishes the effect of IRP1 deletion on the inflammatory process (Example 1, FIG. 1A). In addition, IRP1 deletion significantly reduces joint inflammation typical of rheumatoid arthritis in TNF^{ΔARE/+} mice (Example 2, FIG. 6). These results suggest the use of a selective inhibitor of IRP1 which inhibits activity of expression of IRP1 but does not affect in a significant manner IRP2, for the treatment of an inflammatory disease. Also provided is a novel strategy of identifying inhibitors for specific inhibition of IRP1 (Examples 4-5) as described herein for use along the teachings of the present invention.

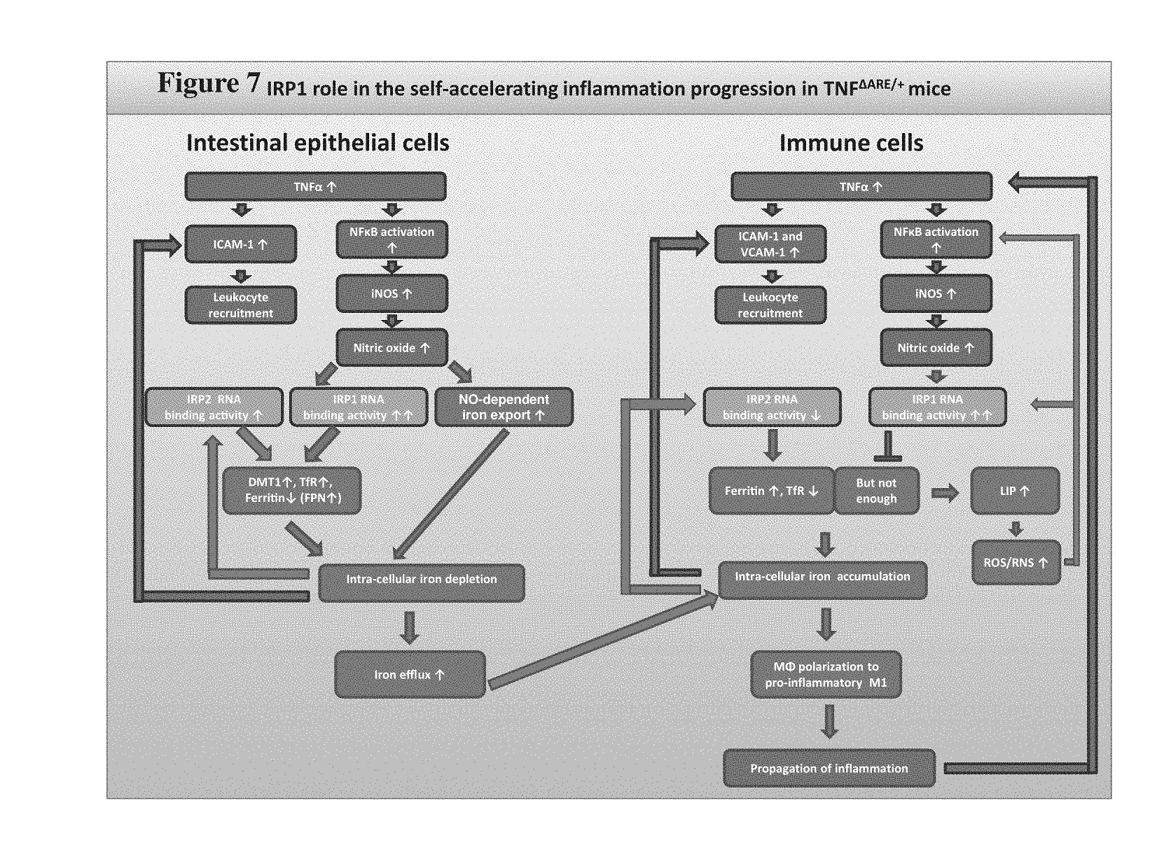

Without being bound by theory, the present inventors suggest (FIG. 7) that inflammation activates IRP1 and IRP1 activation induces a shift of iron stores within the minimally inflamed tissue, which leads to propagation of the inflammation possibly through induction of central immune-system recruitment, induction of ROS/RNS production, which further activates IRP1, and also NFkB pathway which leads to enhanced TNFα and NO production. Thus, without being bound by theory, the beneficial effect of IRP1 deletion and/or inhibition involves a disruption of systemic immune-cell recruitment, consequently inhibiting the expansion of the inflammation.

Thus, according to an aspect of the present invention there is provided a method of treating an inflammatory disease in a subject in need thereof, the method comprising administering to the subject a therapeutically effective amount of an agent which selectively inhibits activity and/or expression of iron regulatory protein (IRP) 1 and not IRP2, thereby treating the inflammatory disease in the subject.

According to another aspect of the present invention there is provided an agent which selectively inhibits activity and/or expression of IRP1 and not IRP2, for use in the treatment of an inflammatory disease in a subject.

The term “treating” refers to inhibiting, preventing or arresting the development of a pathology (disease, disorder, or condition e.g., inflammation e.g., IBD, RA) and/or causing the reduction, remission, or regression of a pathology. Those of skill in the art will understand that various methodologies and assays can be used to assess the development of a pathology, and similarly, various methodologies and assays may be used to assess the reduction, remission or regression of a pathology.

As used herein the phrase “subject in need thereof” refers to a mammalian male or female subject (e.g., human being) who is diagnosed with an inflammatory disease or is at risk of to develop an inflammatory disease. Veterinary uses are also contemplated. The subject may be of any age including neonatal, infant, juvenile, adolescent, adult, elderly adult).

Methods of determining inflammation in a subject are well known in the art and include, but are not limited to, determining in a blood sample from the subject the erythrocyte sedimentation rate (ESR); plasma viscosity; levels of C-reactive protein (CRP); levels of certain inflammatory cytokines such as IL6 and TNFα; and determination of an inflammation index such as using fibrinogen measurements and hematocrit or hemoglobin.

Examples of inflammatory diseases (also referred to herein as inflammation or inflammatory condition) include, but not limited to, chronic inflammatory disease and acute inflammatory disease.

Examples for Inflammatory disease include, but not limited to inflammatory diseases associated with hypersensitivity, autoimmune diseases, infectious diseases, graft rejection diseases, allergic diseases and cancerous diseases.

Inflammatory Diseases Associated with Hypersensitivity

Examples of hypersensitivity include, but are not limited to, Type I hypersensitivity, Type II hypersensitivity, Type III hypersensitivity, Type IV hypersensitivity, immediate hypersensitivity, antibody mediated hypersensitivity, immune complex mediated hypersensitivity, T lymphocyte mediated hypersensitivity and DTH.

Type I or immediate hypersensitivity, such as asthma.

Type II hypersensitivity include, but are not limited to, rheumatoid diseases, rheumatoid autoimmune diseases, rheumatoid arthritis (Krenn V. et al., Histol Histopathol 2000 July; 15 (3):791), spondylitis, ankylosing spondylitis (Jan Voswinkel et al., Arthritis Res 2001; 3 (3): 189), systemic diseases, systemic autoimmune diseases, systemic lupus erythematosus (Erikson J. et al., Immunol Res 1998; 17 (1-2):49), sclerosis, systemic sclerosis (Renaudineau Y. et al., Clin Diagn Lab Immunol. 1999 March; 6 (2):156); Chan O T. et al., Immunol Rev 1999 June; 169:107), glandular diseases, glandular autoimmune diseases, pancreatic autoimmune diseases, diabetes, Type I diabetes (Zimmet P. Diabetes Res Clin Pract 1996 October; 34 Suppl:S125), thyroid diseases, autoimmune thyroid diseases, Graves' disease (Orgiazzi J. Endocrinol Metab Clin North Am 2000 June; 29 (2):339), thyroiditis, spontaneous autoimmune thyroiditis (Braley-Mullen H. and Yu S, J Immunol 2000 Dec. 15; 165 (12):7262), Hashimoto's thyroiditis (Toyoda N. et al., Nippon Rinsho 1999 August; 57 (8):1810), myxedema, idiopathic myxedema (Mitsuma T. Nippon Rinsho. 1999 August; 57 (8):1759); autoimmune reproductive diseases, ovarian diseases, ovarian autoimmunity (Garza K M. et al., J Reprod Immunol 1998 February; 37 (2):87), autoimmune anti-sperm infertility (Diekman A B. et al., Am J Reprod Immunol. 2000 March; 43 (3):134), repeated fetal loss (Tincani A. et al., Lupus 1998; 7 Suppl 2:S107-9), neurodegenerative diseases, neurological diseases, neurological autoimmune diseases, multiple sclerosis (Cross A H. et al., J Neuroimmunol 2001 Jan. 1; 112 (1-2):1), Alzheimer's disease (Oron L. et al., J Neural Transm Suppl. 1997; 49:77), myasthenia gravis (Infante A J. And Kraig E, Int Rev Immunol 1999; 18 (1-2):83), motor neuropathies (Kornberg A J. J Clin Neurosci. 2000 May; 7 (3):191), Guillain-Barre syndrome, neuropathies and autoimmune neuropathies (Kusunoki S. Am J Med Sci. 2000 April; 319 (4):234), myasthenic diseases, Lambert-Eaton myasthenic syndrome (Takamori M. Am J Med Sci. 2000 April; 319 (4):204), paraneoplastic neurological diseases, cerebellar atrophy, paraneoplastic cerebellar atrophy, non-paraneoplastic stiff man syndrome, cerebellar atrophies, progressive cerebellar atrophies, encephalitis, Rasmussen's encephalitis, amyotrophic lateral sclerosis, Sydeham chorea, Gilles de la Tourette syndrome, polyendocrinopathies, autoimmune polyendocrinopathies (Antoine J C. and Honnorat J. Rev Neurol (Paris) 2000 January; 156 (1):23); neuropathies, dysimmune neuropathies (Nobile-Orazio E. et al., Electroencephalogr Clin Neurophysiol Suppl 1999; 50:419); neuromyotonia, acquired neuromyotonia, arthrogryposis multiplex congenita (Vincent A. et al., Ann N Y Acad Sci. 1998 May 13; 841:482), cardiovascular diseases, cardiovascular autoimmune diseases, atherosclerosis (Matsuura E. et al., Lupus. 1998; 7 Suppl 2:S135), myocardial infarction (Vaarala O. Lupus. 1998; 7 Suppl 2:S132), thrombosis (Tincani A. et al., Lupus 1998; 7 Suppl 2:S107-9), granulomatosis, Wegener's granulomatosis, arteritis, Takayasu's arteritis and Kawasaki syndrome (Praprotnik S. et al., Wien Klin Wochenschr 2000 Aug. 25; 112 (15-16):660); anti-factor VIII autoimmune disease (Lacroix-Desmazes S. et al., Semin Thromb Hemost. 2000; 26 (2):157); vasculitises, necrotizing small vessel vasculitises, microscopic polyangiitis, Churg and Strauss syndrome, glomerulonephritis, pauci-immune focal necrotizing glomerulonephritis, crescentic glomerulonephritis (Noel L H. Ann Med Interne (Paris). 2000 May; 151 (3):178); antiphospholipid syndrome (Flamholz R. et al., J Clin Apheresis 1999; 14 (4):171); heart failure, agonist-like beta-adrenoceptor antibodies in heart failure (Wallukat G. et al., Am J Cardiol. 1999 Jun. 17; 83 (12A):75H), thrombocytopenic purpura (Moccia F. Ann Ital Med Int. 1999 April-June; 14 (2):114); hemolytic anemia, autoimmune hemolytic anemia (Efremov D G. et al., Leuk Lymphoma 1998 January; 28 (3-4):285), gastrointestinal diseases, autoimmune diseases of the gastrointestinal tract, intestinal diseases, chronic inflammatory intestinal disease (Garcia Herola A. et al., Gastroenterol Hepatol. 2000 January; 23 (1):16), celiac disease (Landau Y E. and Shoenfeld Y. Harefuah 2000 Jan. 16; 138 (2):122), autoimmune diseases of the musculature, myositis, autoimmune myositis, Sjogren's syndrome (Feist E. et al., Int Arch Allergy Immunol 2000 September; 123 (1):92); smooth muscle autoimmune disease (Zauli D. et al., Biomed Pharmacother 1999 June; 53 (5-6):234), hepatic diseases, hepatic autoimmune diseases, autoimmune hepatitis (Manns M P. J Hepatol 2000 August; 33 (2):326) and primary biliary cirrhosis (Strassburg C P. et al., Eur J Gastroenterol Hepatol. 1999 June; 11 (6):595).

Type IV or T cell mediated hypersensitivity, include, but are not limited to, rheumatoid diseases, rheumatoid arthritis (Tisch R, McDevitt H O. Proc Natl Acad Sci USA 1994 Jan. 18; 91 (2):437), systemic diseases, systemic autoimmune diseases, systemic lupus erythematosus (Datta S K., Lupus 1998; 7 (9):591), glandular diseases, glandular autoimmune diseases, pancreatic diseases, pancreatic autoimmune diseases, Type 1 diabetes (Castano L. and Eisenbarth G S. Ann. Rev. Immunol. 8:647); thyroid diseases, autoimmune thyroid diseases, Graves' disease (Sakata S. et al., Mol Cell Endocrinol 1993 March; 92 (1):77); ovarian diseases (Garza K M. et al., J Reprod Immunol 1998 February; 37 (2):87), prostatitis, autoimmune prostatitis (Alexander R B. et al., Urology 1997 December; 50 (6):893), polyglandular syndrome, autoimmune polyglandular syndrome, Type I autoimmune polyglandular syndrome (Hara T. et al., Blood. 1991 Mar. 1; 77 (5):1127), neurological diseases, autoimmune neurological diseases, multiple sclerosis, neuritis, optic neuritis (Soderstrom M. et al., J Neurol Neurosurg Psychiatry 1994 May; 57 (5):544), myasthenia gravis (Oshima M. et al., Eur J Immunol 1990 December; 20 (12):2563), stiff-man syndrome (Hiemstra H S. et al., Proc Natl Acad Sci USA 2001 Mar. 27; 98 (7):3988), cardiovascular diseases, cardiac autoimmunity in Chagas' disease (Cunha-Neto E. et al., J Clin Invest 1996 Oct. 15; 98 (8):1709), autoimmune thrombocytopenic purpura (Semple J W. et al., Blood 1996 May 15; 87 (10):4245), anti-helper T lymphocyte autoimmunity (Caporossi A P. et al., Viral Immunol 1998; 11 (1):9), hemolytic anemia (Sallah S. et al., Ann Hematol 1997 March; 74 (3):139), hepatic diseases, hepatic autoimmune diseases, hepatitis, chronic active hepatitis (Franco A. et al., Clin Immunol Immunopathol 1990 March; 54 (3):382), biliary cirrhosis, primary biliary cirrhosis (Jones D E. Clin Sci (Colch) 1996 November; 91 (5):551), nephric diseases, nephric autoimmune diseases, nephritis, interstitial nephritis (Kelly C J. J Am Soc Nephrol 1990 August; 1 (2):140), connective tissue diseases, ear diseases, autoimmune connective tissue diseases, autoimmune ear disease (Yoo T J. et al., Cell Immunol 1994 August; 157 (1):249), disease of the inner ear (Gloddek B. et al., Ann N Y Acad Sci 1997 Dec. 29; 830:266), skin diseases, cutaneous diseases, dermal diseases, bullous skin diseases, pemphigus vulgaris, bullous pemphigoid and pemphigus foliaceus.

Examples of delayed type hypersensitivity include, but are not limited to, contact dermatitis and drug eruption.

Examples of types of T lymphocyte mediating hypersensitivity include, but are not limited to, helper T lymphocytes and cytotoxic T lymphocytes.

Examples of helper T lymphocyte-mediated hypersensitivity include, but are not limited to, Tₕ1 lymphocyte mediated hypersensitivity and Tₕ2 lymphocyte mediated hypersensitivity.

Autoimmune Diseases

Include, but are not limited to, cardiovascular diseases, rheumatoid diseases, glandular diseases, gastrointestinal diseases, cutaneous diseases, hepatic diseases, neurological diseases, muscular diseases, nephric diseases, diseases related to reproduction, connective tissue diseases and systemic diseases.

Examples of autoimmune cardiovascular diseases include, but are not limited to atherosclerosis (Matsuura E. et al., Lupus. 1998; 7 Suppl 2:S135), myocardial infarction (Vaarala O. Lupus. 1998; 7 Suppl 2:S132), thrombosis (Tincani A. et al., Lupus 1998; 7 Suppl 2:S107-9), Wegener's granulomatosis, Takayasu's arteritis, Kawasaki syndrome (Praprotnik S. et al., Wien Klin Wochenschr 2000 Aug. 25; 112 (15-16):660), anti-factor VIII autoimmune disease (Lacroix-Desmazes S. et al., Semin Thromb Hemost. 2000; 26 (2):157), necrotizing small vessel vasculitis, microscopic polyangiitis, Churg and Strauss syndrome, pauci-immune focal necrotizing and crescentic glomerulonephritis (Noel L H. Ann Med Interne (Paris). 2000 May; 151 (3):178), antiphospholipid syndrome (Flamholz R. et al., J Clin Apheresis 1999; 14 (4):171), antibody-induced heart failure (Wallukat G. et al., Am J Cardiol. 1999 Jun. 17; 83 (12A):75H), thrombocytopenic purpura (Moccia F. Ann Ital Med Int. 1999 April-June; 14 (2):114; Semple J W. et al., Blood 1996 May 15; 87 (10):4245), autoimmune hemolytic anemia (Efremov D G. et al., Leuk Lymphoma 1998 January; 28 (3-4):285; Sallah S. et al., Ann Hematol 1997 March; 74 (3):139), cardiac autoimmunity in Chagas' disease (Cunha-Neto E. et al., J Clin Invest 1996 Oct. 15; 98 (8):1709) and anti-helper T lymphocyte autoimmunity (Caporossi A P. et al., Viral Immunol 1998; 11 (1):9).

Examples of autoimmune rheumatoid diseases include, but are not limited to rheumatoid arthritis (Krenn V. et al., Histol Histopathol 2000 July; 15 (3):791; Tisch R, McDevitt H O. Proc Natl Acad Sci units S A 1994 Jan. 18; 91 (2):437) and ankylosing spondylitis (Jan Voswinkel et al., Arthritis Res 2001; 3 (3): 189).

Examples of autoimmune glandular diseases include, but are not limited to, pancreatic disease, Type I diabetes, thyroid disease, Graves' disease, thyroiditis, spontaneous autoimmune thyroiditis, Hashimoto's thyroiditis, idiopathic myxedema, ovarian autoimmunity, autoimmune anti-sperm infertility, autoimmune prostatitis and Type I autoimmune polyglandular syndrome. Diseases include, but are not limited to autoimmune diseases of the pancreas, Type 1 diabetes (Castano L. and Eisenbarth G S. Ann. Rev. Immunol. 8:647; Zimmet P. Diabetes Res Clin Pract 1996 October; 34 Suppl:S125), autoimmune thyroid diseases, Graves' disease (Orgiazzi J. Endocrinol Metab Clin North Am 2000 June; 29 (2):339; Sakata S. et al., Mol Cell Endocrinol 1993 March; 92 (1):77), spontaneous autoimmune thyroiditis (Braley-Mullen H. and Yu S, J Immunol 2000 Dec. 15; 165 (12):7262), Hashimoto's thyroiditis (Toyoda N. et al., Nippon Rinsho 1999 August; 57 (8):1810), idiopathic myxedema (Mitsuma T. Nippon Rinsho. 1999 August; 57 (8):1759), ovarian autoimmunity (Garza K M. et al., J Reprod Immunol 1998 February; 37 (2):87), autoimmune anti-sperm infertility (Diekman A B. et al., Am J Reprod Immunol. 2000 March; 43 (3):134), autoimmune prostatitis (Alexander R B. et al., Urology 1997 December; 50 (6):893) and Type I autoimmune polyglandular syndrome (Hara T. et al., Blood. 1991 Mar. 1; 77 (5):1127).

Examples of autoimmune gastrointestinal diseases include, but are not limited to, chronic inflammatory intestinal diseases (Garcia Herola A. et al., Gastroenterol Hepatol. 2000 January; 23 (1):16), celiac disease (Landau Y E. and Shoenfeld Y. Harefuah 2000 Jan. 16; 138 (2):122), colitis, ileitis and Crohn's disease.

Examples of autoimmune cutaneous diseases include, but are not limited to, autoimmune bullous skin diseases, such as, but are not limited to, pemphigus vulgaris, bullous pemphigoid and pemphigus foliaceus.

Examples of autoimmune hepatic diseases include, but are not limited to, hepatitis, autoimmune chronic active hepatitis (Franco A. et al., Clin Immunol Immunopathol 1990 March; 54 (3):382), primary biliary cirrhosis (Jones D E. Clin Sci (Colch) 1996 November; 91 (5):551; Strassburg C P. et al., Eur J Gastroenterol Hepatol. 1999 June; 11 (6):595) and autoimmune hepatitis (Manns M P. J Hepatol 2000 August; 33 (2):326).

Examples of autoimmune neurological diseases include, but are not limited to, multiple sclerosis (Cross A H. et al., J Neuroimmunol 2001 Jan. 1; 112 (1-2):1), Alzheimer's disease (Oron L. et al., J Neural Transm Suppl. 1997; 49:77), myasthenia gravis (Infante A J. And Kraig E, Int Rev Immunol 1999; 18 (1-2):83; Oshima M. et al., Eur J Immunol 1990 December; 20 (12):2563), neuropathies, motor neuropathies (Kornberg A J. J Clin Neurosci. 2000 May; 7 (3):191); Guillain-Barre syndrome and autoimmune neuropathies (Kusunoki S. Am J Med Sci. 2000 April; 319 (4):234), myasthenia, Lambert-Eaton myasthenic syndrome (Takamori M. Am J Med Sci. 2000 April; 319 (4):204); paraneoplastic neurological diseases, cerebellar atrophy, paraneoplastic cerebellar atrophy and stiff-man syndrome (Hiemstra H S. et al., Proc Natl Acad Sci units S A 2001 Mar. 27; 98 (7):3988); non-paraneoplastic stiff man syndrome, progressive cerebellar atrophies, encephalitis, Rasmussen's encephalitis, amyotrophic lateral sclerosis, Sydeham chorea, Gilles de la Tourette syndrome and autoimmune polyendocrinopathies (Antoine J C. and Honnorat J. Rev Neurol (Paris) 2000 January; 156 (1):23); dysimmune neuropathies (Nobile-Orazio E. et al., Electroencephalogr Clin Neurophysiol Suppl 1999; 50:419); acquired neuromyotonia, arthrogryposis multiplex congenita (Vincent A. et al., Ann N Y Acad Sci. 1998 May 13; 841:482), neuritis, optic neuritis (Soderstrom M. et al., J Neurol Neurosurg Psychiatry 1994 May; 57 (5):544) and neurodegenerative diseases.

Examples of autoimmune muscular diseases include, but are not limited to, myositis, autoimmune myositis and primary Sjogren's syndrome (Feist E. et al., Int Arch Allergy Immunol 2000 September; 123 (1):92) and smooth muscle autoimmune disease (Zauli D. et al., Biomed Pharmacother 1999 June; 53 (5-6):234).

Examples of autoimmune nephric diseases include, but are not limited to, nephritis and autoimmune interstitial nephritis (Kelly C J. J Am Soc Nephrol 1990 August; 1 (2):140).

Examples of autoimmune diseases related to reproduction include, but are not limited to, repeated fetal loss (Tincani A. et al., Lupus 1998; 7 Suppl 2:S107-9).

Examples of autoimmune connective tissue diseases include, but are not limited to, ear diseases, autoimmune ear diseases (Yoo T J. et al., Cell Immunol 1994 August; 157 (1):249) and autoimmune diseases of the inner ear (Gloddek B. et al., Ann N Y Acad Sci 1997 Dec. 29; 830:266).

Examples of autoimmune systemic diseases include, but are not limited to, systemic lupus erythematosus (Erikson J. et al., Immunol Res 1998; 17 (1-2):49) and systemic sclerosis (Renaudineau Y. et al., Clin Diagn Lab Immunol. 1999 March; 6 (2):156); Chan O T. et al., Immunol Rev 1999 June; 169:107).

Infectious Diseases

Examples of infectious diseases include, but are not limited to, chronic infectious diseases, subacute infectious diseases, acute infectious diseases, viral diseases, bacterial diseases, protozoan diseases, parasitic diseases, fungal diseases, mycoplasma diseases and prion diseases.

Graft Rejection Diseases

Examples of diseases associated with transplantation of a graft include, but are not limited to, graft rejection, chronic graft rejection, subacute graft rejection, hyper-acute graft rejection, acute graft rejection and graft versus host disease.

Allergic Diseases

Examples of allergic diseases include, but are not limited to, asthma, hives, urticaria, pollen allergy, dust mite allergy, venom allergy, cosmetics allergy, latex allergy, chemical allergy, drug allergy, insect bite allergy, animal dander allergy, stinging plant allergy, poison ivy allergy and food allergy.

Cancerous Diseases

Examples of cancer include but are not limited to carcinoma, lymphoma, blastoma, sarcoma, and leukemia. Particular examples of cancerous diseases but are not limited to: Myeloid leukemia such as Chronic myelogenous leukemia. Acute myelogenous leukemia with maturation. Acute promyelocytic leukemia, Acute nonlymphocytic leukemia with increased basophils, Acute monocytic leukemia. Acute myelomonocytic leukemia with eosinophilia; Malignant lymphoma, such as Burkitt's Non-Hodgkin's; Lymphocytic leukemia, such as Acute lumphoblastic leukemia. Chronic lymphocytic leukemia; Myeloproliferative diseases, such as Solid tumors Benign Meningioma, Mixed tumors of salivary gland, Colonic adenomas; Adenocarcinomas, such as Small cell lung cancer, Kidney, Uterus, Prostate, Bladder, Ovary, Colon, Sarcomas, Liposarcoma, myxoid, Synovial sarcoma, Rhabdomyosarcoma (alveolar), Extraskeletel myxoid chonodrosarcoma, Ewing's tumor; other include Testicular and ovarian dysgerminoma, Retinoblastoma, Wilms' tumor, Neuroblastoma, Malignant melanoma, Mesothelioma, breast, skin, prostate, and ovarian.

According to specific embodiments the inflammatory disease is selected from the group consisting of an autoimmune disease, an infectious disease, cancer and a neurodegenerative disease.

According to specific embodiments the inflammatory disease comprises inflammatory bowel disease (IBD).

As used herein, the phrase “inflammatory bowel disease (IBD)” refers to a group of inflammatory conditions of the colon and small intestine. Non limiting examples include Crohn's disease and ulcerative colitis.

According to other specific embodiments the inflammatory disease comprises rheumatoid arthritis (RA).

As used herein, the phrase “rheumatoid arthritis (RA)” refers to an autoimmune disease which primarily affects the joints. RA includes, but is limited to, adult RA, juvenile iodopathic arthritis, juvenile RA and juvenile chronic arthritis. RA can be diagnosed according to the American Rheumatoid Association criteria for the classification of rheumatoid arthritis, or any similar criteria and includes active, early (active RA diagnosed for at least 8 weeks but no longer than four years) and incipient (polyarthritis that does not fully meet the criteria for a diagnosis of RA, in association with the presence of RA-specific prognostic biomarkers such as anti-CCP and shared epitope) RA.

According to specific embodiments the inflammatory disease is an oxidative stress disease.

As used herein the term “oxidative stress disease” refers to a disease associated with an imbalance between the production of reactive oxygen and the ability to readily detoxify the reactive intermediates or repair the resulting damage. Oxidative stress can damage all components of the cell including DNA, proteins and lipids. It will be appreciated that oxidative stress may be responsible for initiating or otherwise causing the disease. Alternatively, or additionally, the progression of the disease can be affected by any resultant oxidative stress. Non-limiting examples of oxidative stress disease include autoimmune diseases, infection, cancer, diabetes, diabetic vasculopathy, atherosclerosis, heart failure, myocardial infarction, fragile X syndrome, Sickle Cell Disease, lichen planus, vitiligo, autism, chronic fatigue syndrome, cataract, dementia, and neurodegenerative diseases such as Parkinson's disease, Multiple Sclerosis, ALS, multi-system atrophy, Alzheimer's disease, stroke, progressive supranuclear palsy, fronto-temporal dementia with parkinsonism linked to chromosome 17 and Pick's disease.

According to specific embodiments, the inflammatory disease is not Alzheimer's disease.

As used herein, the term “Iron regulatory protein 1 (IRP1)” E.C. 4.2.1.3 also known as cytosolic aconitase, aconitase 1 soluble, cytoplasmic aconitate hydratase, citrate hydro-lyase, ferritin repressor protein and iron-responsive element-binding protein, refers to a polynucleotide and an expression product e.g. protein of the ACO1 gene. According to a specific embodiment, the IRP1 protein refers to the human protein, such as provided in the following GenBank Numbers NP_001265281 and NP_002188.

As used herein, “IRP1” does not refer to the mitochondrial aconitase (also known as Aconitase 2, Mitochondrial, ACO2, Citrate Hydro-Lyase, Aconitate Hydratase, Mitochondrial) which is an enzyme of the citric acid cycle that catalyzes the interconversion of citrate to isocitrate via cis-aconitate.

IRP1 is a bifunctional protein: when cytosolic iron levels are high IRP1 is in a [4Fe-4S] cluster form, which has cytosolic aconitase activity but cannot bind to IREs; while under low iron conditions, the [4Fe-4S] cluster is disassembled, and IRP1 loses its aconitase activity and acquires IRE-binding capacity thereby indirectly increases translation of e.g. transferrin receptor and DMT1 and inhibits translation of e.g. ferritin leading eventually to increased iron uptake and availability.

As used herein, the term “Iron regulatory protein 2 (IRP2)” also known as iron-responsive element binding protein 2, refers to a polynucleotide and an expression product e.g. protein of the IREB2 gene. According to a specific embodiment, the IRP2 protein refers to the human protein, such as provided in the following GenBank Number NP_004127.

IRP-2 does not assemble an Fe—S cluster and spontaneously binds IREs. When iron levels are high, F-box and leucine-rich repeat protein 5 (FBXL5) binds to its target motifs on IRP2 and induces its proteasomal degradation. Under conditions of low iron levels, FBXL5 itself is targeted for ubiquitination and degraded, which stabilizes IRP2 and allows its binding to IREs.

It will be appreciate that selective inhibition of activity and/or expression of IRP1 and not IRP2 can be used to treat an inflammatory disease in the subject. It is contemplated that downregulating the activity and/or expression of IRP2 will augment the inflammatory disease and/or will induce deleterious side effects.

As used herein, the phrases “activity of IRP1” and “activity of IRP2” refers directly to the RNA binding activity or catalytic activity of the protein or to a downstream activity of same. The activities of IRP1 and IRP2 are not shared by the proteins either in a qualitative or quantitative fashion. According to specific embodiments the activity is binding to an iron responsive element (IRE).

IRP family of polypeptides refer to the family of the iron-responsive element-binding proteins, also known as IRE-BP, IRBP, IRP and IFR, that bind to iron-responsive elements in the regulation of iron metabolism.

Examples of IRP targets include but are not limited to FTH1, FTL, TFRC, ALAS2, Sdhb, AC02, Hao1, SLC11A2, NDUFS1, Slc40a1, CDC42BPA, CDC14A, EPAS1.

As used herein, the phrase “iron responsive element (IRE)”, Rfam RF00037, refers to a cis-regulatory nucleic acid motif that interacts with IRPs.

Typically an IRE is a stem and loop structure present in the untranslated regions (UTRs) of a mRNA and binding of IRP to this structure affects post transcriptional regulation of the mRNA. The type of regulation generally depends on the location of the IRE in the target mRNA: binding of IRP1 or IRP2 to the IRE in the 5′ UTR prevents translation, whereas binding to the IRE in the 3′ UTR increases mRNA stability.

As used herein, the phrase “selective inhibition” refers to the ability to specifically downregulate the activity and/or expression of IRP1 and/or conversion of [4Fe-4S] cluster form, which has cytosolic aconitase activity to an IRE binding protein, and not to downregulate the activity and/or expression of IRP2. The selective inhibition can be manifested as higher affinity (e.g., K_{d}) of the agent to one IRP (e.g., IRP1) than to another member of the family (e.g., IRP2). Increased affinity can be of at least 5, 10 or 100 fold.

According to specific embodiments, the “selective inhibition” further refers to the ability to specifically downregulate the activity and/or expression of IRP1 and not to downregulate the activity and/or expression of the mitochondrial aconitase.

Downregulation of IRP can be effected on the genomic and/or the transcript level using a variety of molecules which interfere with transcription and/or translation [e.g., RNA silencing agents (e.g., antisense, siRNA, shRNA, micro-RNA), Ribozyme and DNAzyme], or on the protein level using e.g., small molecules, peptides, antagonists, enzymes that cleave the polypeptide and the like.

Following is a list of agents capable of downregulating expression level and/or activity of IRP, giving IRP1 as an example.

One example, of an agent capable of downregulating an IRP1 is an antibody or antibody fragment capable of specifically binding IRP1. Preferably, the antibody specifically binds at least one epitope of an IRP1. As used herein, the term “epitope” refers to any antigenic determinant on an antigen to which the paratope of an antibody binds.

Epitopic determinants usually consist of chemically active surface groupings of molecules such as amino acids or carbohydrate side chains and usually have specific three dimensional structural characteristics, as well as specific charge characteristics.

The term “antibody” as used in this invention includes intact molecules as well as functional fragments thereof, such as Fab, F(ab′)2, and Fv that are capable of binding to macrophages. These functional antibody fragments are defined as follows: (1) Fab, the fragment which contains a monovalent antigen-binding fragment of an antibody molecule, can be produced by digestion of whole antibody with the enzyme papain to yield an intact light chain and a portion of one heavy chain; (2) Fab′, the fragment of an antibody molecule that can be obtained by treating whole antibody with pepsin, followed by reduction, to yield an intact light chain and a portion of the heavy chain; two Fab′ fragments are obtained per antibody molecule; (3) (Fab′)2, the fragment of the antibody that can be obtained by treating whole antibody with the enzyme pepsin without subsequent reduction; F(ab′)2 is a dimer of two Fab′ fragments held together by two disulfide bonds; (4) Fv, defined as a genetically engineered fragment containing the variable region of the light chain and the variable region of the heavy chain expressed as two chains; and (5) Single chain antibody (“SCA”), a genetically engineered molecule containing the variable region of the light chain and the variable region of the heavy chain, linked by a suitable polypeptide linker as a genetically fused single chain molecule.

Methods of producing polyclonal and monoclonal antibodies as well as fragments thereof are well known in the art (See for example, Harlow and Lane, Antibodies: A Laboratory Manual, Cold Spring Harbor Laboratory, New York, 1988, incorporated herein by reference).

Antibody fragments according to some embodiments of the invention can be prepared by proteolytic hydrolysis of the antibody or by expression in E. coli or mammalian cells (e.g. Chinese hamster ovary cell culture or other protein expression systems) of DNA encoding the fragment. Antibody fragments can be obtained by pepsin or papain digestion of whole antibodies by conventional methods. For example, antibody fragments can be produced by enzymatic cleavage of antibodies with pepsin to provide a 5S fragment denoted F(ab′)2. This fragment can be further cleaved using a thiol reducing agent, and optionally a blocking group for the sulfhydryl groups resulting from cleavage of disulfide linkages, to produce 3.5S Fab′ monovalent fragments. Alternatively, an enzymatic cleavage using pepsin produces two monovalent Fab′ fragments and an Fc fragment directly. These methods are described, for example, by Goldenberg, U.S. Pat. Nos. 4,036,945 and 4,331,647, and references contained therein, which patents are hereby incorporated by reference in their entirety. See also Porter, R. R. [Biochem. J. 73: 119-126 (1959)]. Other methods of cleaving antibodies, such as separation of heavy chains to form monovalent light-heavy chain fragments, further cleavage of fragments, or other enzymatic, chemical, or genetic techniques may also be used, so long as the fragments bind to the antigen that is recognized by the intact antibody.

Fv fragments comprise an association of VH and VL chains. This association may be noncovalent, as described in Inbar et al. [Proc. Nat'l Acad. Sci. USA 69:2659-62 (19720]. Alternatively, the variable chains can be linked by an intermolecular disulfide bond or cross-linked by chemicals such as glutaraldehyde. Preferably, the Fv fragments comprise VH and VL chains connected by a peptide linker. These single-chain antigen binding proteins (sFv) are prepared by constructing a structural gene comprising DNA sequences encoding the VH and VL domains connected by an oligonucleotide. The structural gene is inserted into an expression vector, which is subsequently introduced into a host cell such as E. coli. The recombinant host cells synthesize a single polypeptide chain with a linker peptide bridging the two V domains. Methods for producing sFvs are described, for example, by [Whitlow and Filpula, Methods 2: 97-105 (1991); Bird et al., Science 242:423-426 (1988); Pack et al., Bio/Technology 11:1271-77 (1993); and U.S. Pat. No. 4,946,778, which is hereby incorporated by reference in its entirety.

Another form of an antibody fragment is a peptide coding for a single complementarity-determining region (CDR). CDR peptides (“minimal recognition units”) can be obtained by constructing genes encoding the CDR of an antibody of interest. Such genes are prepared, for example, by using the polymerase chain reaction to synthesize the variable region from RNA of antibody-producing cells. See, for example, Larrick and Fry [Methods, 2: 106-10 (1991)].

Humanized forms of non-human (e.g., murine) antibodies are chimeric molecules of immunoglobulins, immunoglobulin chains or fragments thereof (such as Fv, Fab, Fab′, F(ab′).sub.2 or other antigen-binding subsequences of antibodies) which contain minimal sequence derived from non-human immunoglobulin. Humanized antibodies include human immunoglobulins (recipient antibody) in which residues form a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin [Jones et al., Nature, 321:522-525 (1986); Riechmann et al., Nature, 332:323-329 (1988); and Presta, Curr. Op. Struct. Biol., 2:593-596 (1992)].

Methods for humanizing non-human antibodies are well known in the art. Generally, a humanized antibody has one or more amino acid residues introduced into it from a source which is non-human. These non-human amino acid residues are often referred to as import residues, which are typically taken from an import variable domain. Humanization can be essentially performed following the method of Winter and co-workers [Jones et al., Nature, 321:522-525 (1986); Riechmann et al., Nature 332:323-327 (1988); Verhoeyen et al., Science, 239:1534-1536 (1988)], by substituting rodent CDRs or CDR sequences for the corresponding sequences of a human antibody. Accordingly, such humanized antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

Human antibodies can also be produced using various techniques known in the art, including phage display libraries [Hoogenboom and Winter, J. Mol. Biol., 227:381 (1991); Marks et al., J. Mol. Biol., 222:581 (1991)]. The techniques of Cole et al. and Boerner et al. are also available for the preparation of human monoclonal antibodies (Cole et al., Monoclonal Antibodies and Cancer Therapy, Alan R. Liss, p. 77 (1985) and Boerner et al., J. Immunol., 147(1):86-95 (1991)]. Similarly, human antibodies can be made by introduction of human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in the following scientific publications: Marks et al., Bio/Technology 10: 779-783 (1992); Lonberg et al., Nature 368: 856-859 (1994); Morrison, Nature 368 812-13 (1994); Fishwild et al., Nature Biotechnology 14, 845-51 (1996); Neuberger, Nature Biotechnology 14: 826 (1996); and Lonberg and Huszar, Intern. Rev. Immunol. 13, 65-93 (1995).

Downregulation of IRP1 can be also achieved by RNA silencing. According to a specific embodiment, the agent comprises a siRNA or an antisense oligonucleotides. As used herein, the phrase “RNA silencing” refers to a group of regulatory mechanisms [e.g. RNA interference (RNAi), transcriptional gene silencing (TGS), post-transcriptional gene silencing (PTGS), quelling, co-suppression, and translational repression] mediated by RNA molecules which result in the inhibition or “silencing” of the expression of a corresponding protein-coding gene. RNA silencing has been observed in many types of organisms, including plants, animals, and fungi.

As used herein, the term “RNA silencing agent” refers to an RNA which is capable of specifically inhibiting or “silencing” the expression of a target gene. In certain embodiments, the RNA silencing agent is capable of preventing complete processing (e.g, the full translation and/or expression) of an mRNA molecule through a post-transcriptional silencing mechanism. RNA silencing agents include noncoding RNA molecules, for example RNA duplexes comprising paired strands, as well as precursor RNAs from which such small non-coding RNAs can be generated. Exemplary RNA silencing agents include dsRNAs such as siRNAs, miRNAs and shRNAs. In one embodiment, the RNA silencing agent is capable of inducing RNA interference. In another embodiment, the RNA silencing agent is capable of mediating translational repression.

According to an embodiment of the invention, the RNA silencing agent is specific to the target RNA (e.g., IRP1) and does not cross inhibit or silence a gene or a splice variant which exhibits 99% or less global homology to the target gene, e.g., less than 98%, 97%, 96%, 95%, 94%, 93%, 92%, 91%, 90%, 89%, 88%, 87%, 86%, 85%, 84%, 83%, 82%, 81% global homology to the target gene.

RNA interference refers to the process of sequence-specific post-transcriptional gene silencing in animals mediated by short interfering RNAs (siRNAs). The corresponding process in plants is commonly referred to as post-transcriptional gene silencing or RNA silencing and is also referred to as quelling in fungi. The process of post-transcriptional gene silencing is thought to be an evolutionarily-conserved cellular defense mechanism used to prevent the expression of foreign genes and is commonly shared by diverse flora and phyla. Such protection from foreign gene expression may have evolved in response to the production of double-stranded RNAs (dsRNAs) derived from viral infection or from the random integration of transposon elements into a host genome via a cellular response that specifically destroys homologous single-stranded RNA or viral genomic RNA.

The presence of long dsRNAs in cells stimulates the activity of a ribonuclease III enzyme referred to as dicer. Dicer is involved in the processing of the dsRNA into short pieces of dsRNA known as short interfering RNAs (siRNAs). Short interfering RNAs derived from dicer activity are typically about 21 to about 23 nucleotides in length and comprise about 19 base pair duplexes. The RNAi response also features an endonuclease complex, commonly referred to as an RNA-induced silencing complex (RISC), which mediates cleavage of single-stranded RNA having sequence complementary to the antisense strand of the siRNA duplex. Cleavage of the target RNA takes place in the middle of the region complementary to the antisense strand of the siRNA duplex.

Accordingly, some embodiments of the invention contemplate use of dsRNA to downregulate protein expression from mRNA.

According to one embodiment, the dsRNA is greater than 30 bp. The use of long dsRNAs (i.e. dsRNA greater than 30 bp) has been very limited owing to the belief that these longer regions of double stranded RNA will result in the induction of the interferon and PKR response. However, the use of long dsRNAs can provide numerous advantages in that the cell can select the optimal silencing sequence alleviating the need to test numerous siRNAs; long dsRNAs will allow for silencing libraries to have less complexity than would be necessary for siRNAs; and, perhaps most importantly, long dsRNA could prevent viral escape mutations when used as therapeutics.

Various studies demonstrate that long dsRNAs can be used to silence gene expression without inducing the stress response or causing significant off-target effects—see for example [Strat et al., Nucleic Acids Research, 2006, Vol. 34, No. 13 3803-3810; Bhargava A et al. Brain Res. Protoc. 2004; 13:115-125; Diallo M., et al., Oligonucleotides. 2003; 13:381-392; Paddison P. J., et al., Proc. Natl Acad. Sci. USA. 2002; 99:1443-1448; Tran N., et al., FEBS Lett. 2004; 573:127-134].

In particular, the invention according to some embodiments thereof contemplates introduction of long dsRNA (over 30 base transcripts) for gene silencing in cells where the interferon pathway is not activated (e.g. embryonic cells and oocytes) see for example Billy et al., PNAS 2001, Vol 98, pages 14428-14433. and Diallo et al, Oligonucleotides, Oct. 1, 2003, 13(5): 381-392. doi:10.1089/154545703322617069.

The invention according to some embodiments thereof also contemplates introduction of long dsRNA specifically designed not to induce the interferon and PKR pathways for down-regulating gene expression. For example, Shinagwa and Ishii [Genes & Dev. 17 (11): 1340-1345, 2003] have developed a vector, named pDECAP, to express long double-strand RNA from an RNA polymerase II (Pol II) promoter. Because the transcripts from pDECAP lack both the 5′-cap structure and the 3′-poly(A) tail that facilitate ds-RNA export to the cytoplasm, long ds-RNA from pDECAP does not induce the interferon response.

Another method of evading the interferon and PKR pathways in mammalian systems is by introduction of small inhibitory RNAs (siRNAs) either via transfection or endogenous expression.

The term “siRNA” refers to small inhibitory RNA duplexes (generally between 18-30 basepairs) that induce the RNA interference (RNAi) pathway. Typically, siRNAs are chemically synthesized as 21mers with a central 19 bp duplex region and symmetric 2-base 3′-overhangs on the termini, although it has been recently described that chemically synthesized RNA duplexes of 25-30 base length can have as much as a 100-fold increase in potency compared with 21mers at the same location. The observed increased potency obtained using longer RNAs in triggering RNAi is theorized to result from providing Dicer with a substrate (27mer) instead of a product (21mer) and that this improves the rate or efficiency of entry of the siRNA duplex into RISC.

It has been found that position of the 3′-overhang influences potency of an siRNA and asymmetric duplexes having a 3′-overhang on the antisense strand are generally more potent than those with the 3′-overhang on the sense strand (Rose et al., 2005). This can be attributed to asymmetrical strand loading into RISC, as the opposite efficacy patterns are observed when targeting the antisense transcript.

The strands of a double-stranded interfering RNA (e.g., an siRNA) may be connected to form a hairpin or stem-loop structure (e.g., an shRNA). Thus, as mentioned the RNA silencing agent of some embodiments of the invention may also be a short hairpin RNA (shRNA).

The term “shRNA”, as used herein, refers to an RNA agent having a stem-loop structure, comprising a first and second region of complementary sequence, the degree of complementarity and orientation of the regions being sufficient such that base pairing occurs between the regions, the first and second regions being joined by a loop region, the loop resulting from a lack of base pairing between nucleotides (or nucleotide analogs) within the loop region. The number of nucleotides in the loop is a number between and including 3 to 23, or 5 to 15, or 7 to 13, or 4 to 9, or 9 to 11. Some of the nucleotides in the loop can be involved in base-pair interactions with other nucleotides in the loop. Examples of oligonucleotide sequences that can be used to form the loop include 5′-UUCAAGAGA-3′ (Brummelkamp, T. R. et al. (2002) Science 296: 550) and 5′-UUUGUGUAG-3′ (Castanotto, D. et al. (2002) RNA 8:1454). It will be recognized by one of skill in the art that the resulting single chain oligonucleotide forms a stem-loop or hairpin structure comprising a double-stranded region capable of interacting with the RNAi machinery.

Synthesis of RNA silencing agents suitable for use with some embodiments of the invention can be effected as follows. First, the IRP1 mRNA sequence is scanned downstream of the AUG start codon for AA dinucleotide sequences. Occurrence of each AA and the 3′ adjacent 19 nucleotides is recorded as potential siRNA target sites. Preferably, siRNA target sites are selected from the open reading frame, as untranslated regions (UTRs) are richer in regulatory protein binding sites. UTR-binding proteins and/or translation initiation complexes may interfere with binding of the siRNA endonuclease complex [Tuschl ChemBiochem. 2:239-245]. It will be appreciated though, that siRNAs directed at untranslated regions may also be effective, as demonstrated for GAPDH wherein siRNA directed at the 5′ UTR mediated about 90% decrease in cellular GAPDH mRNA and completely abolished protein level (wwwdotambiondotcom/techlib/tn/91/912dothtml).

Second, potential target sites are compared to an appropriate genomic database (e.g., human, mouse, rat etc.) using any sequence alignment software, such as the BLAST software available from the NCBI server (wwwdotncbidotnlmdotnihdotgov/BLAST/). Putative target sites which exhibit significant homology to other coding sequences are filtered out.

Qualifying target sequences are selected as template for siRNA synthesis. Preferred sequences are those including low G/C content as these have proven to be more effective in mediating gene silencing as compared to those with G/C content higher than 55%. Several target sites are preferably selected along the length of the target gene for evaluation. For better evaluation of the selected siRNAs, a negative control is preferably used in conjunction. Negative control siRNA preferably include the same nucleotide composition as the siRNAs but lack significant homology to the genome. Thus, a scrambled nucleotide sequence of the siRNA is preferably used, provided it does not display any significant homology to any other gene.

For example, a suitable IRP1 siRNA can be the siRNA Cat. No. sc-40713 (Santa-Cruz Biotechnology).

It will be appreciated that the RNA silencing agent of some embodiments of the invention need not be limited to those molecules containing only RNA, but further encompasses chemically-modified nucleotides and non-nucleotides.

In some embodiments, the agent provided herein can be functionally associated with a cell-penetrating peptide.” As used herein, a “cell-penetrating peptide” is a peptide that comprises a short (about 12-30 residues) amino acid sequence or functional motif that confers the energy-independent (i.e., non-endocytotic) translocation properties associated with transport of the membrane-permeable complex across the plasma and/or nuclear membranes of a cell. The cell-penetrating peptide used in the membrane-permeable complex of some embodiments of the invention preferably comprises at least one non-functional cysteine residue, which is either free or derivatized to form a disulfide link with a double-stranded ribonucleic acid that has been modified for such linkage. Representative amino acid motifs conferring such properties are listed in U.S. Pat. No. 6,348,185, the contents of which are expressly incorporated herein by reference. The cell-penetrating peptides of some embodiments of the invention preferably include, but are not limited to, penetratin, transportan, pIs1, TAT(48-60), pVEC, MTS, and MAP.

Another agent capable of downregulating an IRP1 is a DNAzyme molecule capable of specifically cleaving an mRNA transcript or DNA sequence of the IRP1. DNAzymes are single-stranded polynucleotides which are capable of cleaving both single and double stranded target sequences (Breaker, R. R. and Joyce, G. Chemistry and Biology 1995; 2:655; Santoro, S. W. & Joyce, G. F. Proc. Natl, Acad. Sci. USA 1997; 943:4262) A general model (the “10-23” model) for the DNAzyme has been proposed. “10-23” DNAzymes have a catalytic domain of 15 deoxyribonucleotides, flanked by two substrate-recognition domains of seven to nine deoxyribonucleotides each. This type of DNAzyme can effectively cleave its substrate RNA at purine:pyrimidine junctions (Santoro, S. W. & Joyce, G. F. Proc. Natl, Acad. Sci. USA 199; for rev of DNAzymes see Khachigian, L M [Curr Opin Mol Ther 4:119-21 (2002)].

Examples of construction and amplification of synthetic, engineered DNAzymes recognizing single and double-stranded target cleavage sites have been disclosed in U.S. Pat. No. 6,326,174 to Joyce et al. DNAzymes of similar design directed against the human Urokinase receptor were recently observed to inhibit Urokinase receptor expression, and successfully inhibit colon cancer cell metastasis in vivo (Itoh et al, 20002, Abstract 409, Ann Meeting Am Soc Gen Ther wwwdotasgtdotorg). In another application, DNAzymes complementary to bcr-ab1 oncogenes were successful in inhibiting the oncogenes expression in leukemia cells, and lessening relapse rates in autologous bone marrow transplant in cases of CML and ALL.

Downregulation of a IRP1 can also be effected by using an antisense polynucleotide capable of specifically hybridizing with an mRNA transcript encoding the IRP.

Design of antisense molecules which can be used to efficiently downregulate an IRP1 must be effected while considering two aspects important to the antisense approach. The first aspect is delivery of the oligonucleotide into the cytoplasm of the appropriate cells, while the second aspect is design of an oligonucleotide which specifically binds the designated mRNA within cells in a way which inhibits translation thereof.

The prior art teaches of a number of delivery strategies which can be used to efficiently deliver oligonucleotides into a wide variety of cell types [see, for example, Luft J Mol Med 76: 75-6 (1998); Kronenwett et al. Blood 91: 852-62 (1998); Rajur et al. Bioconjug Chem 8: 935-40 (1997); Lavigne et al. Biochem Biophys Res Commun 237: 566-71(1997) and Aoki et al. (1997) Biochem Biophys Res Commun 231: 540-5 (1997)].

In addition, algorithms for identifying those sequences with the highest predicted binding affinity for their target mRNA based on a thermodynamic cycle that accounts for the energetics of structural alterations in both the target mRNA and the oligonucleotide are also available [see, for example, Walton et al. Biotechnol Bioeng 65: 1-9 (1999)].

Such algorithms have been successfully used to implement an antisense approach in cells. For example, the algorithm developed by Walton et al. enabled scientists to successfully design antisense oligonucleotides for rabbit beta-globin (RBG) and mouse tumor necrosis factor-alpha (TNF alpha) transcripts. The same research group has more recently reported that the antisense activity of rationally selected oligonucleotides against three model target mRNAs (human lactate dehydrogenase A and B and rat gp130) in cell culture as evaluated by a kinetic PCR technique proved effective in almost all cases, including tests against three different targets in two cell types with phosphodiester and phosphorothioate oligonucleotide chemistries.

In addition, several approaches for designing and predicting efficiency of specific oligonucleotides using an in vitro system were also published (Matveeva et al., Nature Biotechnology 16: 1374-1375 (1998)].

Another agent capable of downregulating an IRP1 is a ribozyme molecule capable of specifically cleaving an mRNA transcript encoding an mRNA. Ribozymes are being increasingly used for the sequence-specific inhibition of gene expression by the cleavage of mRNAs encoding proteins of interest [Welch et al., Curr Opin Biotechnol. 9:486-96 (1998)]. The possibility of designing ribozymes to cleave any specific target RNA has rendered them valuable tools in both basic research and therapeutic applications. In the therapeutics area, ribozymes have been exploited to target viral RNAs in infectious diseases, dominant oncogenes in cancers and specific somatic mutations in genetic disorders [Welch et al., Clin Diagn Virol. 10:163-71 (1998)]. Most notably, several ribozyme gene therapy protocols for HIV patients are already in Phase 1 trials. More recently, ribozymes have been used for transgenic animal research, gene target validation and pathway elucidation. Several ribozymes are in various stages of clinical trials. ANGIOZYME was the first chemically synthesized ribozyme to be studied in human clinical trials. ANGIOZYME specifically inhibits formation of the VEGF-r (Vascular Endothelial Growth Factor receptor), a key component in the angiogenesis pathway. Ribozyme Pharmaceuticals, Inc., as well as other firms have demonstrated the importance of anti-angiogenesis therapeutics in animal models. HEPTAZYME, a ribozyme designed to selectively destroy Hepatitis C Virus (HCV) RNA, was found effective in decreasing Hepatitis C viral RNA in cell culture assays (Ribozyme Pharmaceuticals, Incorporated—WEB home page).

It will be appreciated that a non-functional analogue of at least a catalytic or binding portion of IRP1 can be also used as an agent which downregulates IRP1.

Another agent which can be used along with some embodiments of the invention to downregulate IRP1 is a molecule which prevents IRP1 activation or substrate binding.

According to specific embodiments the agent is a small molecule or a peptide which affect the interaction of IRP1 with a nucleic acid sequence (i.e. mRNA) containing IRE.

Agents resulting from various screening methods which can be used according to the present teachings are also contemplated herein.

Screening methods are known in the art and have been described for example in Xavier et al. [Trends Biotech., (2000) 18: 349-356] and Ecker and Giffey [Drug Discov Today. (1999) 4(9):420-429], Tibodeau et al. [PNAS (2006) 103 (2): 253-257], US Publication No. US20110281744 and U.S. Pat. No. 7,078,171, each of which is incorporated herein by reference.

Alternatively or additionally the present teachings are directed to the identification of compounds as according to the following aspect.

Thus, according to another aspect of the present invention there is provided a method of identifying an agent that selectively modulates an activity of an IRP member of an IRP family of polypeptides and not of an additional IRP member of the IRP family of polypeptides, the method comprising:
(a) determining an effect of a test agent on binding of the IRP member to at least one nucleic acid sequence containing an IRE and/or on expression of a reporter gene comprising the at least one nucleic acid sequence containing the IRE; and
(b) determining an effect of the test agent on binding of the additional IRP member to at least one nucleic acid sequence containing the IRE and/or on expression of the reporter gene comprising the at least one nucleic acid sequence containing the IRE; wherein:
(i) an alteration in binding of the IRP member to the at least one nucleic acid sequence containing the IRE and/or expression of the reporter gene comprising the at least one nucleic acid sequence containing the IRE nucleic acid sequence as compared to same in an absence of the test agent; and
(ii) no alteration in binding of the additional IRP member to the at least one nucleic acid sequence containing the IRE and/or expression of the reporter gene comprising the at least one nucleic acid sequence containing the IRE as compared to same in an absence of the test agent,
are indicative of an agent that selectively modulates activity of an IRP member of an IRP family of polypeptides and not of an additional IRP member of the IRP family of polypeptides.

The methods of the present invention can be contemplated both in identifying an agent that selectively modulates activity and/or expression of IRP1 and not IRP2 and to an agent that selectively modulates activity and/or expression of IRP2 and not IRP1.

According to specific embodiments, the IRP member is IRP1 and the additional IRP member is IRP2.

According to other specific embodiments, the IRP member is IRP2 and the additional IRP member is IRP1.

As used herein the phrase “nucleic acid sequence containing an IRE” refers to a single or double stranded nucleic acid sequence that contains a cis-regulatory IRE.

According to a specific embodiment, the at least one nucleic acid sequence containing said IRE comprises at least two, at least three or more different nucleic acid sequences containing the IRE.

As used herein, the term “modulates” refers to altering IRP activity either by inhibiting or by activating.

According to specific embodiments, modulates activity is inhibits activity.

According to specific embodiments, modulates activity is activates activity.

As used herein, the term “altering” or “alteration” refers to a change in the level of binding of IRP to a nucleic acid sequence containing an IRE as measured by a change in the binding itself and/or in the level of expression of a reporter gene operatively connected to a nucleic acid containing the IRE. The change can be either a decrease or increase.

According to specific embodiments the change is at least 1.5 fold, at least 2 fold, at least 3 fold, at least 5 fold, at least 10 fold, or at least 20 fold.

As used herein, the term “binding” refers to the interaction of IRP with an IRE. The binding can be evaluated per se (e.g., binding affinity e.g., using plasmon resonance BIAcore assay) or by determining the effect of binding on expression of a reporter gene translationally fused to the nucleic acid sequence containing the IRE. Generally, binding of an IRP to an IRE affects post-transcriptional regulation of the nucleic acid sequence containing the IRE wherein the regulation depends on the location of the IRE in the target nucleic acid sequence. Typically, as disclosed above, binding of either IRPs to the IRE in the 5′ UTR (i.e upstream of the reporter gene) prevents translation, whereas binding to the IRE in the 3′ UTR (i.e. downstream of the reported gene) increases mRNA stability.

Depending on the location of the IRE, the effect on expression of the reporter gene can be either upregulation or downregulation of expression.

According to specific to embodiments the effect on expression of all of the nucleic acid sequences containing said IRE is upregulation; downregulation; or upregulation of at least one and downregulation of at least one.

Thus, depending on the assay the reporter gene can be positioned upstream or downstream of the nucleic acid sequence containing the IRE. Measures are taken to retain the regulatory effect of the IRE on the transcription of the reporter gene.

According to a specific embodiment, the nucleic acid sequence containing said IRE is positioned upstream of said reporter gene.

According to another embodiment, nucleic acid sequence containing said IRE is positioned downstream of said reporter gene.

Thus, the location of the reporter gene can vary e.g. all of the nucleic acid sequences containing said IRE are positioned upstream of the reporter gene, all of the nucleic acid sequences containing said IRE are positioned downstream of the reporter gene; or one of the at least two different nucleic acid sequences containing said IRE is positioned upstream of the reporter gene and the second of the at least two different nucleic acid sequences containing said IRE is positioned downstream of said reporter gene.

According to a specific embodiment, one of the at least two different nucleic acid sequences containing said IRE is positioned upstream of the reporter gene and the second of the at least two different nucleic acid sequences containing said IRE is positioned downstream of said reporter gene.

Binding is also affected by multiple exogenous conditions such as iron concentration, oxygen concentration, nitrosative stress and oxidative stress. For example, low iron concentration increases biding activity of the IRPs to the IREs, while high iron concentration decreases binding activity of the IRPs to the IREs. Thus for example, under low iron concentration an IRP inhibitor will increase expression of a reporter gene fused to a nucleic acid sequence containing the IRE in the 5′ UTR and will decrease expression of a reporter gene fused to a nucleic acid sequence containing the IRE in the 3′ UTR.

According to specific embodiments, the method is effected under low iron conditions.

According to specific embodiments, the method is effected under high iron conditions.

According to other specific embodiments, the method is effected under oxidative and/or nitrosative stress conditions.

According to specific embodiments, the reporter gene comprising a nucleic acid sequence containing the IRE can be naturally occurring molecule or a chimeric molecule.

According to a specific embodiment, the reporter gene comprising a nucleic acid sequence containing the IRE is a naturally occurring molecule.

As used herein, the phrase “naturally occurring molecule” refers to an mRNA containing an IRE in its UTR which is found in nature. Non limiting examples of a naturally occurring molecule that can be used in the methods of the present invention are transferrin receptor, ferritin and DMT1. Thus, according to a specific embodiment, the nucleic acid sequence containing said IRE is of a polynucleotide selected from the group consisting of transferrin receptor IRE, ferritin IRE, and DMT1 IRE. According to specific embodiments, the naturally occurring molecule can be a full mRNA sequence or a fragment thereof. According to specific embodiment, the naturally occurring molecule is comprised, either endogenously or exogenously, in a cell.

According to another specific embodiment, the reporter gene comprising a nucleic acid sequence containing the IRE is a chimeric molecule.

As used herein, the phrase “chimeric molecule” refers to induced synthetic molecule comprising a nucleic acid sequence containing an IRE and a reporter gene which are heterologous. According to a specific embodiment, the chimeric molecule is comprised in a cell.

According to specific embodiments, the reporter gene comprises (attached or conjugated to) a detectable moiety.

According to specific embodiments, determining the effect of a test agent on binding of IRP to a nucleic acid sequence containing an IRE and/or on expression of a reporter gene comprising a nucleic acid sequence containing an IRE comprises the detection of the detectable moiety.

Various types of detectable moieties may be conjugated to the nucleic acid containing an IRE. According to specific embodiment the detectable moiety is a translational product. These include, but not are limited to, a phosphorescent chemical, a hemiluminescent chemical such as luciferase and galactosidase, a fluorescent chemical (fluorophore) such as GFP, an enzyme, a fluorescent polypeptide, an affinity tag, and molecules (contrast agents) detectable by Positron Emission Tomagraphy (PET) or Magnetic Resonance Imaging (MRI).

Fluorescence detection methods which can be used to detect the expression of the nucleic acid containing an IRE when conjugated to a fluorescent detectable moiety include, for example, fluorescent plate reader, fluorescence activated flow cytometry (FACS), immunofluorescence confocal microscopy, fluorescence in-situ hybridization (FISH) and fluorescence resonance energy transfer (FRET).

Non limiting example of a chemiluminescent chemical is luciferase. Chemiluminescent detection methods which can be used to detect the expression of the nucleic acid containing an IRE when conjugated to a chemiluminescent moiety include, for example, luminescence plate reader.

Detection of the detectable moiety can be effected by methods and apparatuses well known in the art including, but not limited to flow cytometer, fluorescent plate reader and luminescence plate reader.

To test the differential effect of an agent on one of the IRPs and not the other the method can utilize cells not expressing one of the IPRs e.g. cells stably knocked out of either IRP2 (IRP2−/−) or IRP1 (IRP1−/−).

Thus, according to a specific embodiment, the cell does not express endogenous IRP1 and/or IRP2.

According to a specific embodiment one of steps (a) or (b) is effected in a cell not expressing one of the IRPs endogenously (e.g. IRP1) and the other is effected in a cell not expressing the second IRP endogenously (e.g. IRP2).

Non-limiting examples of cell lines that can be used in the present invention include Jurkat, CEM, THP1, Caco-2, EBV-immortalized B-cells from primary donors and the like.

Alternatively or additionally, the cell line used can be a cell line which is not viable in the absence of both IRP1 and IRP2 and stably knock out IRP2 (IRP2−/−) or IRP1 (IRP1−/−) in this line. Thus, an IRP1 inhibitor induces death of the IRP2−/− cells and an inhibitor of IRP2 induces death of the IRP1−/− cells. Methods of monitoring viability are known in the art and include for example, the MTT test which is based on the selective ability of living cells to reduce the yellow salt MTT (3-(4, 5-dimethylthiazolyl-2)-2, 5-diphenyltetrazolium bromide) (Sigma-Aldrich St Louis, Mo., USA) to a purple-blue insoluble formazan precipitate; the BrDu assay [Cell Proliferation ELISA BrdU colorimetric kit (Roche, Mannheim, Germany]; the TUNEL assay [Roche, Mannheim, Germany]; the Annexin V assay [ApoAlert® Annexin V Apoptosis Kit (Clontech Laboratories, Inc., CA, USA)]; and propidium iodide (PI) staining (Sigma-Aldrich).

Once suitable agents are identified they are synthesized and may be further qualified using immune cell models such as macrophage cell model and/or animal models of inflammation such as disclosed hereinbelow. Agents which qualify under the predetermined screens are qualified as suitable for the treatment of inflammation.

While further reducing the present invention to practice, the present inventors have further devised in-silico screening tools which can be used to identify agents, which qualify for use according to some embodiments of the invention.

Thus, according to an aspect of the invention, there is provided a method of identifying an agent that selectively inhibits an activity of an IRP1, the method comprising:

(a) in-silico selecting a test agent that inhibits binding of an RNA binding form of IRP1 to an IRE but does not inhibit of IRP2 to an IRE; or

(b) in-silico selecting a test agent stabilizing a 4Fe-4S cluster form of IRP1.

Specific embodiments of the method are described in Example 5 hereinbelow which (being a theoretical example) is to be understood as forming an integral part of the present section.

Agents identified accordingly, are further qualified by providing the test agent and testing an anti-inflammatory activity of same.

Such assays are well known in the art and described in details in Example 6 of the instant application which is to be understood as forming an integral part of the present section.

For increasing robustness, lowering the costs such a qualification is effected in-vitro. Alternatively or additionally, in vivo testing for anti-inflammatory activity may be performed.

Any of the above-agents of the invention can be administered to an organism per se, or in a pharmaceutical composition where it is mixed with suitable carriers or excipients.

Thus, according to another aspect of the present invention there is provided a pharmaceutical composition comprising, as an active ingredient, an agent which selectively inhibits activity and/or expression of IRP1 and not IRP2, and a pharmaceutically acceptable carrier or excipient.

As used herein a “pharmaceutical composition” refers to a preparation of one or more of the active ingredients described herein with other chemical components such as physiologically suitable carriers and excipients. The purpose of a pharmaceutical composition is to facilitate administration of a compound to an organism.

Herein the term “active ingredient” refers to the agent accountable for the biological effect, i.e. inhibition of the activity and/or expression of IRP1 and not IRP2.

Hereinafter, the phrases “physiologically acceptable carrier” and “pharmaceutically acceptable carrier” which may be interchangeably used refer to a carrier or a diluent that does not cause significant irritation to an organism and does not abrogate the biological activity and properties of the administered compound. An adjuvant is included under these phrases.

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences,” Mack Publishing Co., Easton, Pa., latest edition, which is incorporated herein by reference.

Suitable routes of administration may, for example, include oral, rectal, transmucosal, especially transnasal, intestinal or parenteral delivery, including intramuscular, subcutaneous and intramedullary injections as well as intrathecal, direct intraventricular, intracardiac, e.g., into the right or left ventricular cavity, into the common coronary artery, intravenous, intraperitoneal, intranasal, or intraocular injections.

Conventional approaches for drug delivery to the central nervous system (CNS) include: neurosurgical strategies (e.g., intracerebral injection or intracerebroventricular infusion); molecular manipulation of the agent (e.g., production of a chimeric fusion protein that comprises a transport peptide that has an affinity for an endothelial cell surface molecule in combination with an agent that is itself incapable of crossing the BBB) in an attempt to exploit one of the endogenous transport pathways of the BBB; pharmacological strategies designed to increase the lipid solubility of an agent (e.g., conjugation of water-soluble agents to lipid or cholesterol carriers); and the transitory disruption of the integrity of the BBB by hyperosmotic disruption (resulting from the infusion of a mannitol solution into the carotid artery or the use of a biologically active agent such as an angiotensin peptide). However, each of these strategies has limitations, such as the inherent risks associated with an invasive surgical procedure, a size limitation imposed by a limitation inherent in the endogenous transport systems, potentially undesirable biological side effects associated with the systemic administration of a chimeric molecule comprised of a carrier motif that could be active outside of the CNS, and the possible risk of brain damage within regions of the brain where the BBB is disrupted, which renders it a suboptimal delivery method.

Alternately, one may administer the pharmaceutical composition in a local rather than systemic manner, for example, via injection of the pharmaceutical composition directly into a tissue region of a patient.

The term “tissue” refers to part of an organism consisting of cells designed to perform a function or functions. Examples include, but are not limited to, brain tissue, retina, skin tissue, hepatic tissue, pancreatic tissue, bone, cartilage, connective tissue, blood tissue, muscle tissue, cardiac tissue brain tissue, vascular tissue, renal tissue, pulmonary tissue, gonadal tissue, hematopoietic tissue.

Pharmaceutical compositions of some embodiments of the invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with some embodiments of the invention thus may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

For injection, the active ingredients of the pharmaceutical composition may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

For oral administration, the pharmaceutical composition can be formulated readily by combining the active compounds with pharmaceutically acceptable carriers well known in the art. Such carriers enable the pharmaceutical composition to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions, which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by nasal inhalation, the active ingredients for use according to some embodiments of the invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in a dispenser may be formulated containing a powder mix of the compound and a suitable powder base such as lactose or starch.

The pharmaceutical composition described herein may be formulated for parenteral administration, e.g., by bolus injection or continuous infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily or water based injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the active ingredients to allow for the preparation of highly concentrated solutions.

Alternatively, the active ingredient may be in powder form for constitution with a suitable vehicle, e.g., sterile, pyrogen-free water based solution, before use.

The pharmaceutical composition of some embodiments of the invention may also be formulated in rectal compositions such as suppositories or retention enemas, using, e.g., conventional suppository bases such as cocoa butter or other glycerides.

Pharmaceutical compositions suitable for use in context of some embodiments of the invention include compositions wherein the active ingredients are contained in an amount effective to achieve the intended purpose. More specifically, a therapeutically effective amount means an amount of active ingredients effective to prevent, alleviate or ameliorate symptoms of a disorder (e.g., inflammatory disease) or prolong the survival of the subject being treated.

Determination of a therapeutically effective amount is well within the capability of those skilled in the art, especially in light of the detailed disclosure provided herein.

For any preparation used in the methods of the invention, the therapeutically effective amount or dose can be estimated initially from in vitro and cell culture assays. For example, a dose can be formulated in animal models to achieve a desired concentration or titer. Such information can be used to more accurately determine useful doses in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p. 1).

Dosage amount and interval may be adjusted individually to provide levels of the active ingredient that are sufficient to induce or suppress the biological effect (minimal effective concentration, MEC). The MEC will vary for each preparation, but can be estimated from in vitro data. Dosages necessary to achieve the MEC will depend on individual characteristics and route of administration. Detection assays can be used to determine plasma concentrations.

Depending on the severity and responsiveness of the condition to be treated, dosing can be of a single or a plurality of administrations, with course of treatment lasting from several days to several weeks or until cure is effected or diminution of the disease state is achieved.

The amount of a composition to be administered will, of course, be dependent on the subject being treated, the severity of the affliction, the manner of administration, the judgment of the prescribing physician, etc.

Animal models of IBD include but are not limited to TNF^{ΔARE/+} mice [Kontoyiannis et al. Immunity (1999) 10(3):387-98] and trinitrobenzene sulfonic acid (TNBS)-induced colitis in rats and mice [Komori et al., J Gastroenterol (2005) 40: 591-599]. An animal model of RA includes but is not limited to TNF^{ΔARE/+} mice [Kontoyiannis et al. Immunity (1999) 10(3):387-98] An animal model for adjuvant arthritis (AA, a model of rheumatoid arthritis) includes the rat heat-killed Mt strain H37Ra-induced AA [Kannan, Theor Biol Med Model. (2005) 2:17]. An animal model for asthma includes the Ovalbumin (OVA) sensitization mouse model [Flaishon, L., et al., J. Immunol: Cutting edge 168: 3707 (2002)].

Compositions of the present invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising a preparation of the invention formulated in a compatible pharmaceutical carrier may also be prepared, placed in an appropriate container, and labeled for treatment of an indicated condition, as is further detailed above.

According to specific embodiments, the agent of the present invention can be used alone or in combination with other established or experimental therapeutic regimen to treat an inflammatory disease.

Anti-inflammatory agents which may be used according to the present teachings include, but are not limited to, Alclofenac; Alclometasone Dipropionate; Algestone Acetonide; Alpha Amylase; Amcinafal; Amcinafide; Amfenac Sodium; Amiprilose Hydrochloride; Anakinra; Anirolac; Anitrazafen; Apazone; Balsalazide Disodium; Bendazac; Benoxaprofen; Benzydamine Hydrochloride; Bromelains; Broperamole; Budesonide; Carprofen; Cicloprofen; Cintazone; Cliprofen; Clobetasol Propionate; Clobetasone Butyrate; Clopirac; Cloticasone Propionate; Cormethasone Acetate; Cortodoxone; Deflazacort; Desonide; Desoximetasone; Dexamethasone Dipropionate; Diclofenac Potassium; Diclofenac Sodium; Diflorasone Diacetate; Diflumidone Sodium; Diflunisal; Difluprednate; Diftalone; Dimethyl Sulfoxide; Drocinonide; Endrysone; Enlimomab; Enolicam Sodium; Epirizole; Etodolac; Etofenamate; Felbinac; Fenamole; Fenbufen; Fenclofenac; Fenclorac; Fendosal; Fenpipalone; Fentiazac; Flazalone; Fluazacort; Flufenamic Acid; Flumizole; Flunisolide Acetate; Flunixin; Flunixin Meglumine; Fluocortin Butyl; Fluorometholone Acetate; Fluquazone; Flurbiprofen; Fluretofen; Fluticasone Propionate; Furaprofen; Furobufen; Halcinonide; Halobetasol Propionate; Halopredone Acetate; Ibufenac; Ibuprofen; Ibuprofen Aluminum; Ibuprofen Piconol; Ilonidap; Indomethacin; Indomethacin Sodium; Indoprofen; Indoxole; Intrazole; Isoflupredone Acetate; Isoxepac; Isoxicam; Ketoprofen; Lofemizole Hydrochloride; Lomoxicam; Loteprednol Etabonate; Meclofenamate Sodium; Meclofenamic Acid; Meclorisone Dibutyrate; Mefenamic Acid; Mesalamine; Meseclazone; Methylprednisolone Suleptanate; Momiflumate; Nabumetone; Naproxen; Naproxen Sodium; Naproxol; Nimazone; Olsalazine Sodium; Orgotein; Orpanoxin; Oxaprozin; Oxyphenbutazone; Paranyline Hydrochloride; Pentosan Polysulfate Sodium; Phenbutazone Sodium Glycerate; Pirfenidone; Piroxicam; Piroxicam Cinnamate; Piroxicam Olamine; Pirprofen; Prednazate; Prifelone; Prodolic Acid; Proquazone; Proxazole; Proxazole Citrate; Rimexolone; Romazarit; Salcolex; Salnacedin; Salsalate; Sanguinarium Chloride; Seclazone; Sermetacin; Sudoxicam; Sulindac; Suprofen; Talmetacin; Talniflumate; Talosalate; Tebufelone; Tenidap; Tenidap Sodium; Tenoxicam; Tesicam; Tesimide; Tetrydamine; Tiopinac; Tixocortol Pivalate; Tolmetin; Tolmetin Sodium; Triclonide; Triflumidate; Zidometacin; Zomepirac Sodium.

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

When reference is made to particular sequence listings, such reference is to be understood to also encompass sequences that substantially correspond to its complementary sequence as including minor sequence variations, resulting from, e.g., sequencing errors, cloning errors, or other alterations resulting in base substitution, base deletion or base addition, provided that the frequency of such variations is less than 1 in 50 nucleotides, alternatively, less than 1 in 100 nucleotides, alternatively, less than 1 in 200 nucleotides, alternatively, less than 1 in 500 nucleotides, alternatively, less than 1 in 1000 nucleotides, alternatively, less than 1 in 5,000 nucleotides, alternatively, less than 1 in 10,000 nucleotides.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., Eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Materials and Methods

Mice

TNF^{ΔARE/+} mice on C57BL/6 background [Kontoyiannis et al. Immunity (1999) 10(3):387-98] were used as IBD model. TNF^{ΔARE/+} on IRP1 knockout background (TNF^{ΔARE/+} IRP1−/−) were produced by crossing TNF^{ΔARE/+} mice with mice carrying a targeted deletion of IRP1 [Meyron-Holtz et al. EMBO J. (2004) 23(2):386-95]. F1 TNF^{ΔARE/+ IRP1+/-} mice were crossed again with IRP1^{-/-} mice to yield TNF^{ΔARE/+ IRP1-/-} mice. TNF^{ΔARE/+} heterozygous for IRP1 deletion were produced by crossing TNF^{ΔARE/+} mice with IRP1^{-/-} mice. TNF^{ΔARE/+} on IRP2 knockout (TNF^{ΔARE/+}IRP2−/−) were produced by crossing TNF^{ΔARE/+} mice with mice carrying a targeted deletion of IRP2 (LaVaute T. et al. Nat Genet. (2001) 27(2)209-214). TNF^{ΔARE/+} on IRP1 knockout background and heterozygous for IRP2 deletion were produced by crossing TNF^{ΔARE/+} IRP1−/− with IRP1 knockout mice that are also heterozygous for IRP2 deletion.

The tip of a tail from crossed mice of weaning age 21-28 days was removed and lysed in DirectPCR Lysis Reagent (Viagen) according to manufacturer's instruction. Genotypes were confirmed by PCR amplification. TNFα gene allelic composition was identified with the following primers: 5′-CTT CCT CAC AGA GCC AGC-3′ (SEQ ID NO: 1) forward primer and 5′-GATGCAGACTTCATCCCAAGA-3′ (SEQ ID NO: 2) reverse primer giving 400 bp band for wild type and 500 bp for ΔARE mutant. IRP1 knockout was identified with the following primers: 5′-AGCTCATTCCTCCACTCATG-3′ (SEQ ID NO: 3) and 5′ ACAGACACAGATGCCAGAGG-3′ (SEQ ID NO: 4) forward primers and 5′-GCATGCATCCATTGTCTCTG-3′ (SEQ ID NO: 5) reverse primer giving 350 bp and 450 bp bands representing wild type and knockout allele, respectively. IRP2 knockout was identified with the following primers: 5′-ACGTGTCCTGTTTGCCCTTGTATC-3′ (SEQ ID NO: 6) and 5′ TCTGTAAAGAGTGGTCCACTGTGAGX-3′ (SEQ ID NO: 7) forward primers and 5′-CAGCCTCTGTTCCACATACACTTC-3′ (SEQ ID NO: 8) reverse primer giving 569 bp and 627 bp bands representing wild type and knockout allele, respectively.

Iron Overload—

To generate iron overload in mice, 12-13 week-old mice were injected intra-peritoneally with a total of 45 mg iron in the form of iron-dextran: 100 μl iron-dextran in saline (90 mg iron/ml) five days a week for one week. Mice were sacrificed three days after the last injection for further evaluation.

Histological Evaluation—

Mice were sacrificed at 12-14 weeks of age. The terminal ileum (5 cm) was dissected and parts of it (0.5 cm) were fixed in 4% PFA solution. The tissues were paraffin embedded approximately 20 hr following fixation, slides were prepared and stained with Hematoxylin and Eosin (H&E), iron stain, or immunofluorescent L-ferrtin. Immunofluorescent staining was performed with an antibody against mouse L-ferrtin (a gift from Prof. M. Konijn, Hebrew University of Jerusalem) and DAPI nuclear staining. Staining with only secondary antibody served as negative control. The terminal ileum samples were subjected to histology evaluation in a semi-quantitative blind manner by an experimental and toxicological pathologist, which used a five levels grading score (0, normal; 1, minimal; 2, mild; 3, moderate; 4, severe) taking into consideration the degree of severity and the type of lesion. The joints were histologically examined as previously reported [Kontoyiannis et al. Immunity (1999) 10(3):387-98].

Western Blot and Electrophoretic Mobility Shift Assay—

Mice were sacrificed at 12-14 weeks of age. For determining the levels of proteins involved in iron homeostasis, the terminal ileum (5 cm) was dissected and Intestinal epithelial cells (IEC) enriched fraction was obtained as previously described [Zigmond E. et al. Immunity (2012) 37:1076-1090] in 3% 02. Alternatively, for all tissue analysis the terminal ileum was snap-frozen in liquid N2. The samples were lysed in lysis buffer consisting of 10 mM HEPES (pH 7.2), 3 mM MgCl2, 40 mM KCl, 5% glycerol, 0.2% Nonidet P-40, 5 mM DTT, 1 mM AEBSF, 10 mg/ml Leupeptin and Complete™ EDTA free protease inhibitor cocktail (Roche Applied Science, Indiana). Samples were subsequently subjected to Electrophoretic mobility shift assay for IRP1 activity and Western blot analysis as previously described [Meyron-Holtz et al. EMBO J. (2004) 23:386-95 and Leichtmann-Bardoogo Y. et al. Am J Physiol Endocrinol Metab. (2012) 302(12): 1519-30] with antibodies against different proteins involved in iron homeostasis and inflammation: TNF-α (Abcam, cat# ab1793), IRP2, L-Ferritin, and Ferropprtin (a kind gift from Tracey Rouault), DMT1 (Abnova, cat# H00004891MO1), Transferrin receptor 1 (Abcam, cat# ab84036), and β-Actin (Santacruz, sc-1616 1:2500).

Quantitative RT-PCR

TNF-α mRNA levels in the terminal ileum of the mice were evaluated by qRT-PCR by the following procedure: RNA was extracted from the terminal ileum with TRIZOL reagent (Invitrogen, cat#: 15596-018) according to the manufacturer's protocol. To avoid any genomic DNA in RNA samples, all RNA samples were submitted to DNase treatment using DNase I recombinant, RNase-free kit (Roche, cat#: 04716728001). One microgram of total RNA was reverse transcribed to cDNA using High Capacity cDNA Reverse Transcriptase kit (Ambion, Austin, Tex., USA) according to the manufacturer's protocol. Real-time RT-PCR analysis was performed as previously described [Khalfin-Rabinovich Y. et al. Int. Immunol. (2011) 23 (4): 287-296] by using the following primers: murin TNF-α forward AGACCCTCACACTCAGATCATCTTCT (SEQ ID NO: 9), TNF-α reverse CTGCTCCTCCACTTGGTGGT (SEQ ID NO: 10), β-actin forward AGCCTTCCTTCTTGGGTATGG (SEQ ID NO: 11), and β-actin reverse TCAACGTCACACTTCATGATGG (SEQ ID NO: 12). The estimated amount of transcripts was normalized to β-actin mRNA expression. The data are presented as the relative expression of the gene of interest compared with β-actin.

### Example 1

### IRP1 Knockout Prevents Intestinal Inflammation in a Inflammatory Bowel Disease (IBD) Mouse Model

Absence of IRP1 Prevents Intestinal Inflammation in TNF^{ΔARE/+} Mice while the Presence of IRP2 is Necessary to Avoid Inflammation

TNF^{ΔARE/+} mice over-express TNF-α due to a deletion of a regulatory motif in the 3′UTR of the gene, resulting in increased TNF-α transcription [Kontoyiannis, D. et al. (1999) Immunity 10, 387-398] and severe intestinal inflammation. In the first step intestinal inflammation was confirmed in the TNF^{ΔARE/+} mice. To this end, TNF^{ΔARE/+} and healthy C57Bl/6 wild type (wt) 12-14 weeks old mice were sacrificed and the inflammation was evaluated in histological terminal ileum tissue sections. The representative histological images (FIG. 1A) show that the TNF^{ΔARE/+} mice suffer from severe transmural intestinal inflammation. Similarly, TNF^{ΔARE/+} mice on IRP2^{-/-} background suffer from severe transmural intestinal inflammation (FIG. 1C). On the contrary, both IRP1^{-/-} and TNF^{ΔARE/+} mice on IRP1^{-/-} background, that were sacrificed at the same age show no inflammation pathology. This dramatic effect is significantly emphasized by the low total histological inflammation score of the ileum sections in all three wt, IRP1^{-/-} and TNF^{ΔARE/+} on IRP1^{-/-} background mice relatively to TNF^{ΔARE/+} mice (p<0.0001) (FIG. 1B). Most importantly, the dramatic effect of IRP1 deletion on the inflammatory process was completely abolished in the TNFΔARE/+IRP1−/−IRP2+/− mice. These results significantly indicate specifically inhibiting IRP1 and not IRP2 for the treatment of IBD.

Absence of IRP1 Attenuates the Alteration in Iron Homeostasis During the Intestinal Inflammation Process in TNF^{ΔARE/+} Mice

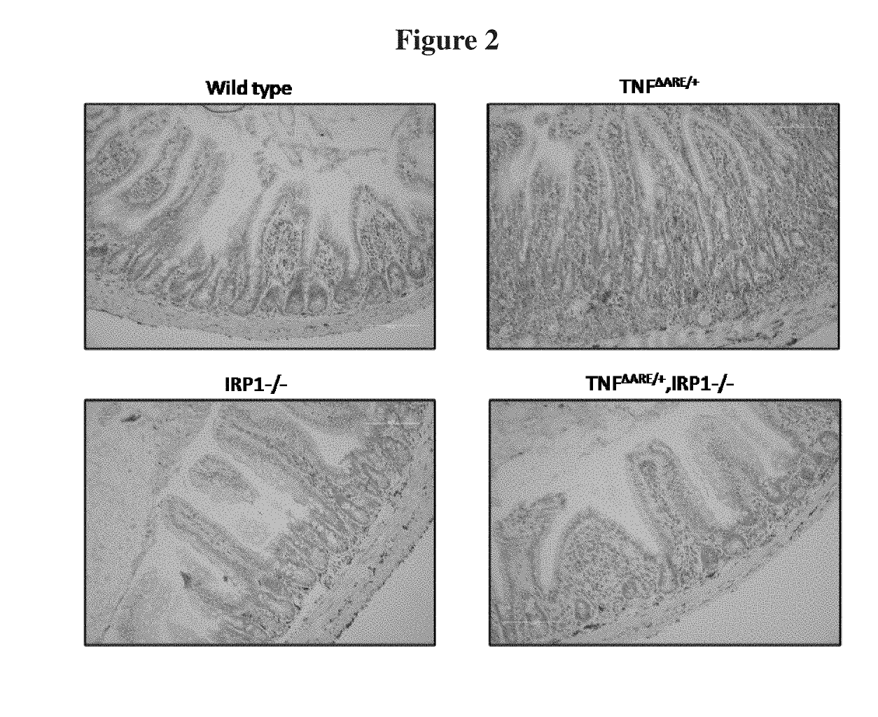

Since iron accumulation in the inflamed area might be a consequence of the inflammation and also play an important role in the inflammatory process iron accumulation was tested in the TNF^{ΔARE/+} mice and compared to iron accumulation in the TNF^{ΔARE/+} mice on IRP1^{-/-} background. Representative iron stained histological sections (FIG. 2) show that iron levels are elevated only in the inflamed intestine of TNF^{ΔARE/+} mice and that the iron accumulation is indeed not present in the TNF^{ΔARE/+} mice on IRP1^{-/-} background. This iron accumulation is mainly detected in the immune cells infiltrating the inflamed area and not in the epithelial cells, thus the low iron levels in the TNF^{ΔARE/+} on IRP1^{-/-} background mice seems to be mainly due to a reduction of the immune cells density in the intestinal section.

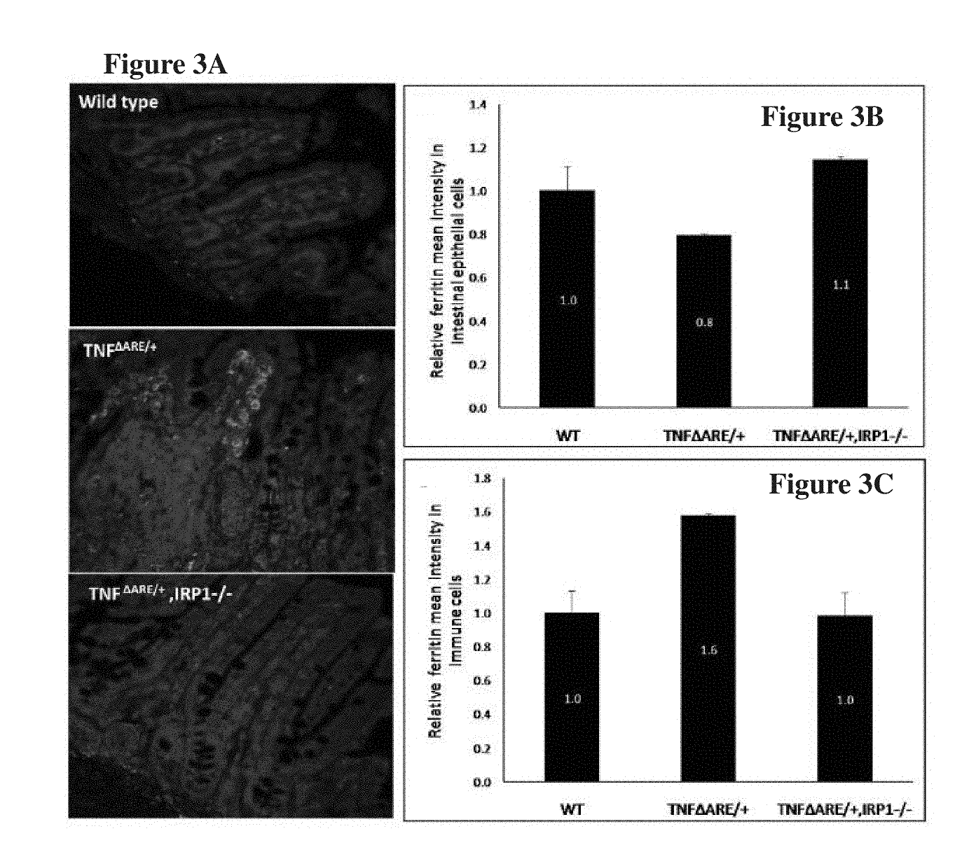

The observed effect on iron accumulation was further verified by determining ferritin levels in the TNF^{ΔARE/+} mice as compared to TNF^{ΔARE/+} mice on IRP1^{-/-} background. Usually, intracellular ferritin levels are proportional to the intracellular iron levels. As can be seen in FIGS. 3A-C, during the inflammation in the TNF^{ΔARE/+} mice there is a significant altered iron redistribution, including decreased iron level in the intestinal epithelial cells (IEC) and iron accumulation in the lamina propria (LP) immune cells. Also evident is the normal iron distribution in the TNF^{ΔARE/+} mice on IRP1^{-/-} background, which suggests that in the absence of IRP1, IRP2 is able to properly regulate iron homeostasis.

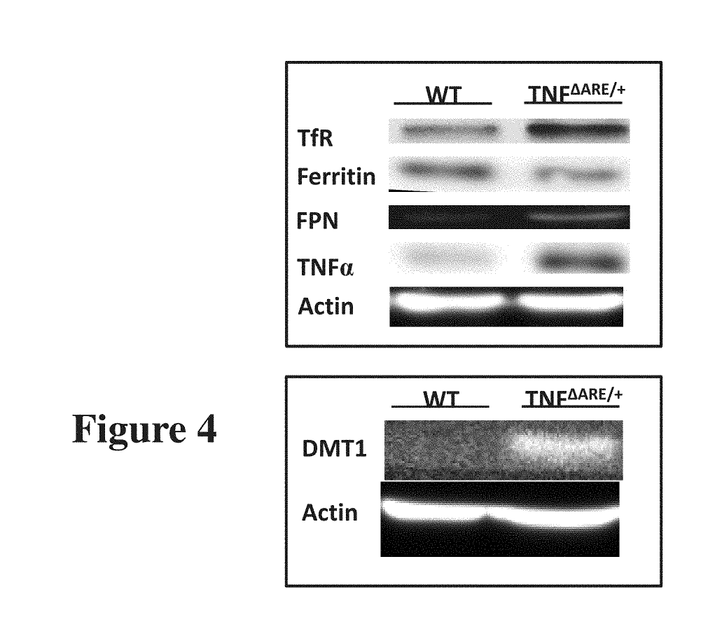

As shown in FIG. 4, expression levels of the different proteins involved in iron homeostasis are significantly altered in the IECs of TNF^{ΔARE/+} mice. Specifically, the elevated IRP1 level is accompanied by elevated levels of IRP2 and the transcriptional factor, Hypoxia Inducible Factor (HIF)-2α. Due to the elevated IRPs activity ferritin levels are decreased and the iron import via TfR is increased. The iron export is increased via FPN, which is upregulated due to the elevated HIF-2α levels. These results indicate that the IECs absorb iron from the blood and export the iron to their surrounding, where it is scavenged by the adjacent immune cells. This leads to an iron relocation which can explain the observed iron decrease in the IEC and iron increase in the LP immune cells.

Absence of IRP1 Prevents TNFα Overexpression in the Terminal Ileum of TNF^{ΔARE/+} Mice

TNF^{ΔARE/+} mice over-express TNF-α due to a deletion of a regulatory motif in the 3′UTR of the gene, resulting in increased TNF-α transcription [Kontoyiannis, D. et al. (1999) Immunity 10, 387-398]. To test whether the IRP1 deletion influences TNF-α expression, the TNF-α protein and mRNA levels were determined by Western blot analysis and by qRT-PCR, respectively.

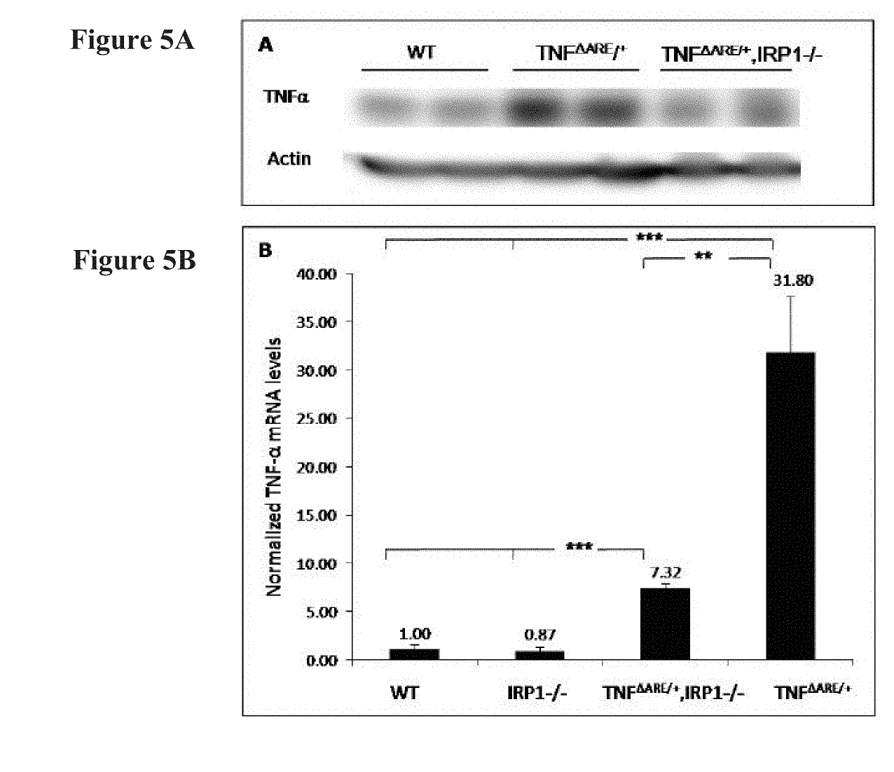

TNF^{ΔARE/+} mice on IRP1^{-/-} background were found to express significantly lower amounts of both TNF-α mRNA and protein in comparison to the TNF^{ΔARE/+} (see FIGS. 5A-B). These relatively reduced TNF-α transcription and subsequently low TNF-α protein levels induced by the IRP1 deletion may explain the effect of IRP1 on inflammation.

### Example 2

### IRP1 Knock Out Reduced Inflammation in Joint in a Rheumatois Arthritis (RA) Mouse Model

The results presented suggest that IRP1 is involved in inflammatory diseases in which iron homeostasis is altered in general and not restricted to a specific type or model. Thus, in order to establish the generality of these findings the involvement of IRP1 in rheumatoid arthritis (RA) was evaluated. RA is a systemic autoimmune disorder characterized by chronic inflammation in joint tissues leading to destruction, deformity, and loss of function of the joint.

TNF^{ΔARE/+} mice present joint inflammation typical of RA and thus, this model was utilized in order to study the role of IRP1 in the pathogenesis of RA. The joints of the TNF^{ΔARE/+} mice on IRP1−/− background were analyzed and compared to TNF^{ΔARE/+} mice heterozygous for IRP1 joints. As can be seen in FIG. 6, TNF^{ΔARE/+} mice heterozygous for IRP1 suffer from severe synovial inflammation, whereas TNF^{ΔARE/+} mice on IRP1−/− background show a significantly reduced inflammatory phenotype.

### Example 3

### Proposed Mechanism of Action of IRP1 Deletion and/or Inhibition on Inflamation

Without being bound by theory, trying to elucidate the mechanism of action of IRP1 knock out on inflammation in general and intestinal inflammation in the TNF^{ΔARE/+} mice model the present inventors suggest the following mechanism (see FIG. 7):

TNF-α, in the immune cells, induces the nuclear factor-kappa B (NFκB) pathway, which is involved in the regulation of many inflammation-associated genes, including inducible nitric oxide synthase (iNOS)[Aktan F (2004) Life sciences 75(6):639-653]. iNOS produces nitric oxide (NO) that is secreted from the immune cells and then subsequently absorbed by the adjacent IECs. In the IECs, the NO activates IRP1 RNA-binding activity. This non iron mediated activation of IRP1 plays a key role in the iron redistribution within the inflammatory lesion. The IRP1 activation fools the cell into an iron deficiency mode and mediates increased iron uptake into the IEC from the bloodstream, through elevated TfR and elevated DMT1. In parallel, the IECs also export more iron through elevated ferroportin. In addition, the NO causes accelerated iron export, resulting in decreased intracellular iron levels, and thus in elevated RNA-binding activity of IRP2. In the literature, it is documented that in IECs low iron levels can cause elevated Mitogen-activated protein kinases (MAPK) activity [Choi, E.-Y. et al. (2004). J. Immunol. Baltim. Md. 1950 172, 7069-7077; and Markel, T. A. et al. (2007) Am. J. Physiol. Gastrointest. Liver Physiol. 292, G958-963]. The elevated MAPK activity can result in pro-inflammatory cytokines production and in elevated levels of additional pro-inflammatory substances such as Inter-cellular adhesion molecule-1 (ICAM-1) and the pro-inflammatory cytokine IL-8 [YAN Wen-sheng et al. (2002) Chin J Pathophysiol 18 (9): 1029-1033; and Choi, E.-Y. et al. (2004). J. Immunol. Baltim. Md. 1950 172, 7069-7077]. IECs are important regulators of the innate and adaptive immunity, therefore this pro-inflammatory effect can result in local leukocyte activation and peripheral leukocyte recruitment. Simultaneously, the exported iron is scavenged by the adjacent phagocytic immune cells resulting in elevated intra-cellular iron levels in the LP immune cells. The iron accumulation in the local immune cells can also cause elevated MAPK activity and elevated reactive oxygen and nitrogen (ROS/RNS) production through the Fenton reaction. This will results in:
1. Macrophage polarization to the pro-inflammatory M1 phenotype [Kroner A, et al. (2014) Neuron 83(5):1098-1116].
2. ROS/RNS can damage biological membranes, because elevated membrane permeability and even cell-death by ferroptosis [Halliwell B (1994) Lancet 344(8924):721-724; and Dixon S J, et al. (2012) Cell 149(5):1060-1072].
3. Additional elevated levels of ICAM-1 and leukocyte recruitment [Martin-Malo A, et al. (2012) Nephrology, dialysis, transplantation: official publication of the European Dialysis and Transplant Association—European Renal Association 27(6):2465-2471; and Cartee T V, et al. (2012) Journal of dermatological science 65(2):86-94].

In the immune-cells, these NO-induced alternations cause a unique scenario in which IRP1 is activated by the NO, while IRP2 levels are decreased due to the elevated iron levels. Meaning that IRP1 interferes with IRP2 proper regulation of iron homeostasis.

Moreover, the combined downstream effects of the inflammation-induced elevated IRP1-RNA binding activity results in self-aggravation of the inflammation through three main loops:
1. Iron accumulation induces central immune-system recruitment through ICAM.
2. Iron accumulation induces ROS/RNS production, which further activates IRP1, which further interferes with proper iron regulation through IRP2.
3. ROS/RNS production activates the NFkB pathway directly [Xiong, S. et al. (2003). J. Biol. Chem. 278, 17646-17654], which leads to enhanced TNFα and NO production and increases IRP1-RNA binding activity.

In summary, the deletion of the TNF^{ARE} induces a local inflammation that activates IRP1, despite normal iron levels. IRP1 activation induces a shift of iron stores within the minimally inflamed tissue, which leads to propagation of the inflammation possibly through the 3 loops mentioned above. Thus, without being bound by theory, the beneficial effect of IRP1 deletion and/or inhibition involves a disruption of systemic immune-cell recruitment, consequently inhibiting the expansion of the inflammation.

Taken together, IRP1 is a master regulator of inflammatory propagation and its inhibition can be used for the suppression of a range of inflammatory diseases such as IBD and RA. Moreover, the data emphasize the significance of the specific inhibition of IRP1 and not IRP2 for the treatment of inflammatory diseases.

### Example 4

### Identifying Candidates for Specific Inhibition of IRP1

To find an agent that specifically affects IRP1 or IRP2 a high throughput assay is effected using a human cell-line (hCS) in a suspension culture. Suggested lines include Jurkat-cells which comprise a stable knockout IRP2 (IRP2−/−) or IRP1 (IRP1−/−). In each of these cells a luciferase containing reporter gene, expressing luciferase under the tyrosine kinase promoter, a medium strength promoter is stably inserted.

To test for inhibitors, luciferase expression is regulated by an IRE located at the 5′ UTR, such as the 5′ IRE of human L-ferritin. When these cells are grown at low iron condition, endogenous IRP binding to IREs is high and therefore luciferase production is inhibited by the binding of IRP1 or IRP2 to the 5′ IRE. In the presence of a specific inhibitor for IRP1 or IRP2, the inhibition of luciferase production is suppressed, and luciferase is produced only in one of the two cell lines that contain the IRP of which RNA binding activity is inhibited. Alternatively, luciferase expression is regulated by IREs located in the 3′ UTR, such as the five IREs at the 3′ of human transferrin receptor (TfR). Under low iron conditions, IRP binding to IREs is high and therefore luciferase is being produced at high amounts by the binding of IRP1 or IRP2 to the 3′ IRE. In the presence of a specific inhibitor for IRP1 or IRP2, the luciferase production is suppressed only in one of the two cell lines that contains the IRP of which RNA binding activity is inhibited.

To test for activators, luciferase expression is regulated by IREs located in the 3′ UTR, such as the five IREs at the 3′ of human TfR. When these cells are grown at high iron condition, endogenous IRP binding to IREs is low and therefore luciferase production is low due to endonucleases that access this part of the mRNA and initiate its degradation. In the presence of a specific activator for IRP1 or IRP2, the degradation of luciferase mRNA is suppressed, and luciferase is produced only in one of the two cell lines that contains the IRP of which RNA binding activity is activated. Alternatively, luciferase expression is regulated by an IRE located in the 5′ UTR, such as the 5′ IRE of human L-ferritin. Under high iron conditions IRP binding to IREs is low and therefore luciferase is produced at high amounts. In the presence of a specific activator for IRP1 or IRP2, luciferase production is inhibited only in one of the two cell lines that contains the IRP of which RNA binding activity is activated.

Luciferase production is determined using luminescence plate reader.

Alternatively or additionally, the reporter gene is a fluorescent reporter gene and the fluorophore expression is determined using flow cytometry or fluorescent plate reader. The use of fluorescent reporter genes allows using two constructs with different fluorophores one with a 5′ UTR IRE and another with a 3′ UTR IRE thereby the two constructs are regulated by IRP in two different directions.

Alternatively or additionally, the cell line used is a cell line which is not viable in the absence of both IRP1 and IRP2 and stably knock out IRP2 (IRP2−/−) or IRP1 (IRP1−/−) in this line. Thus, an IRP1 inhibitor induces death of the IRP2−/− cells and an inhibitor of IRP2 induces death of the IRP1−/− cells. Methods of monitoring viability are known in the art and include for example, the MTT test which is based on the selective ability of living cells to reduce the yellow salt MTT (3-(4, 5-dimethylthiazolyl-2)-2, 5-diphenyltetrazolium bromide) (Sigma-Aldrich St Louis, Mo., USA) to a purple-blue insoluble formazan precipitate; the BrDu assay [Cell Proliferation ELISA BrdU colorimetric kit (Roche, Mannheim, Germany]; the TUNEL assay [Roche, Mannheim, Germany]; the Annexin V assay [ApoAlert® Annexin V Apoptosis Kit (Clontech Laboratories, Inc., CA, USA)]; and propidium iodide (PI) staining (Sigma-Aldrich).

As IRPs activity is also affected by factors such as oxygen and nitrogen stress the screen is performed under oxidative or nitrosative stress conditions.

The screen is done either in single concentration or in multiple doses for dose-dependent activity.

The selected agents are used for further studies, for example in a macrophage cell model.

### Example 5

### Identifying Candidates for Specific Inhibition of IRP1 Using Rational Drug Design

Specific inhibition of IRP1 is effected using computer guided screening of molecules, based on analyzing and applying molecular properties, molecular interactions and their physical basis, as well as the relation between molecular properties and their specific biological activities.

Several crystal structures of IRP1 and related proteins are known, that provide the foundation for a structure based computational effort to discover small molecule inhibitors of the IRP-1:m-RNA interaction [4, 5]

The search for drug candidates implements two approaches:

A. Direct inhibition of the interaction of the RNA binding form of IRP1 with the IRE.

B. Indirect inhibition, by stabilizing the 4Fe-4S cluster of cytosolic aconitase, to prevent or restrict the conversion of aconitase to IRP1.

Both approaches need to be selective: one is directed specifically to the RNA binding by IRP-1, to prevent its binding to the relevant IRE-mRNA structure, but should not inhibit the ability of IRP-2 to interact with IRE-structures. The second approach is directed to cytosolic aconitase and has to be distinguished from the mitochondrial aconitase that must not be inhibited. All the screenings use commercially available databases of molecules, which contain overall about 20 million molecules.

A. Pharmacophore search for candidate IRP-1 inhibitors: The H-bonds between the IRE and the IRP protein [6] are used to form a set of “vectors” defined by their directions and approximate lengths, including the relative positions of those vectors vis-à-vis each other. Such a set of H-donors and H-acceptors forms a “pharmacophore” which is used to search the databases for molecules that could “mimic” parts of the IRE binding to IRP1 based on their close overlap with the “pharmacophore”.

Selected screened molecules are further qualified using docking and an evaluation of the free energy of complex formation. The main test is “docking”, in which those top molecules are driven, each to interact with the 3-dimensional structure of IRP1. The criteria of successful docking are mainly those of the energy gain in forming the complex, as well as the number of protein residues found to be in contact (H-bonds or Van der Waals contacts) for each of the candidate inhibitors. Further criteria for selection of promising molecules include evaluations of molecular solubility and toxicities, for which the Goldblum group has developed working models, some of which were already published [7, 8]. A small library of the 25-50 most promising molecules is purchased and tested for biological activity. The results are used to refine the pharmacophore model by further criteria, and a second screening takes place.

B. Ligand mimics for aconitase inhibition/stabilization: Combines the pharmacophore approach with a search for mimics of citrate. The pharmacophore approach may be readily applied as it is clear from the crystal structure (1C96 in the Protein Data Bank www.rcsb.org) that citrate forms a very intricate set of hydrogen bonds with the aconitase residues that are close to the 4Fe-4S cluster. Filling part of these H-bonds by “citrate mimics” could be the best basis to search for candidate inhibitors of the citrate interaction with the Fe—S cluster. Selection of the most promising molecules, testing and refinement is applied as described above for IRP1 inhibitors.

Iterative Stochastic Elimination (ISE): In order to refine the selection and discover more effective drug candidates, ISE is be applied to the selection process, utilizing feedback from the biological drug testing. This algorithm from the Goldblum group has been used with exceptional success for discovering molecular bioactive molecules [9]. The core of this algorithm is geared to distinguish between two groups of molecules (“classification”) by creating “filters” of molecular properties, picking the optimal ranges of properties out of an enormous number of possibilities. In most cases, it is applied to distinguish between known active molecules and inactive or weakly active ones.

### Example 6

### Biological Testing of Candidate Molecules

Candidate molecules selected according to Example 4 and Example 5 are tested for their IRP1 inhibitory activity and specificity and for their toxicity. In addition, anti-inflammatory properties are analyzed in a co-culture model for proof of principle. In the first round, 50-100 candidate IRP1 inhibitory molecules are screened. Initially, inhibitory activity and specificity for IRP1 and not IRP2 is tested in Caco-2 cells using electromobility shift assays (EMSA)[10]. Specificity for IRP1 versus IRP2 can easily be analyzed in this assay. For determination of specificity for cytosolic aconitase and not mitochondrial aconitase, a metabolic assay is used. Cells are be grown at a low glucose concentration, shifting cells toward mitochondrial energy production, which requires mitochondrial aconitase. Cross-inhibition of this enzyme is done by simply analyzing cell viability with Alamar blue or MTT reagents, both evaluating mitochondrial function. For anti-inflammatory screens an in vitro cell-based model of intestinal mucosa is used [11]. This model closely mimics tissue complexity and allows to look at combined effects on several cell types simultaneously. To assess the effect of IRP1 inhibitors on inflammation, an inflammatory state is induced in the co-culture and test the effect of candidate molecules on inflammatory markers such as TNFα and IL-8 at mRNA and protein levels.

### REFERENCES FOR EXAMPLES 5-6

1. Frearson, J. A. and I. T. Collie, HTS and hit finding in academia—from chemical genomics to drug discovery. Drug Discov Today, 2009. 14(23-24): p. 1150-8.
2. Talele, T. T., S. A. Khedkar, and A. C. Rigby, Successful applications of computer aided drug discovery: moving drugs from concept to the clinic. Curr Top Med Chem, 2010. 10(1): p. 127-41.
3. Groenhof, G., Solving chemical problems with a mixture of quantum-mechanical and molecular mechanics calculations: Nobel Prize in Chemistry 2013. Angew Chem Int Ed Engl, 2013. 52(48): p. 12489-91.
4. Selezneva, A. I., W. E. Walden, and K. W. Volz, Nucleotide-specific recognition of iron-responsive elements by iron regulatory protein 1. J Mol Biol, 2013. 425(18): p. 3301-10.
5. Dupuy, J., et al., Crystal structure of human iron regulatory protein 1 as cytosolic aconitase. Structure, 2006. 14(1): p. 129-39.
6. Walden, W. E., et al., Structure of dual function iron regulatory protein 1 complexed with ferritin IRE-RNA. Science, 2006. 314(5807): p. 1903-8.
7. Cern, A., et al., Computer-aided design of liposomal drugs: In silico prediction and experimental validation of drug candidates for liposomal remote loading. J Control Release, 2014. 173: p. 125-31.
8. Rayan, A., D. Marcus, and A. Goldblum, Predicting oral druglikeness by iterative stochastic elimination. J Chem Inf Model, 2010. 50(3): p. 437-45.
9. Stern, N. and A. Goldblum, Iterative Stochastic Elimination for Solving Complex Combinatorial Problems in Drug Discovery. Israel Journal of Chemistry, 2014. 54(8-9): p. 1338-1357.
10. Meyron-Holtz, E. G., M. C. Ghosh, and T. A. Rouault, Mammalian tissue oxygen levels modulate iron-regulatory protein activities in vivo. Science, 2004. 306(5704): p. 2087-90.
11. Leonard, F., E. M. Collnot, and C. M. Lehr, A three-dimensional coculture of enterocytes, monocytes and dendritic cells to model inflamed intestinal mucosa in vitro. Mol Pharm, 2010. 7(6): p. 2103-19.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1. A method of treating an inflammatory disease in a subject in need thereof, the method comprising administering to the subject a therapeutically effective amount of an agent which selectively inhibits activity and/or expression of iron regulatory protein (IRP) 1 and not IRP2, thereby treating the inflammatory disease in the subject.

2. (canceled)

3. A pharmaceutical composition comprising, as an active ingredient, an agent which selectively inhibits activity and/or expression of IRP1 and not IRP2, and a pharmaceutically acceptable carrier or excipient.

4. The method of claim 1, wherein said activity is binding to an iron responsive element (IRE).

5. The method of claim 1, wherein said inflammatory disease is selected from the group consisting of an autoimmune disease, an infectious disease, cancer and a neurodegenerative disease.

6. The method of claim 1, wherein said inflammatory disease comprises inflammatory bowel disease (IBD).

7. The method of claim 1, wherein said inflammatory disease comprises rheumatoid arthritis (RA).

8. The method of claim 1, wherein said inflammatory disease is an oxidative stress disease.

9. (canceled)

10. A method of identifying an agent that selectively inhibits an activity of an IRP1, the method comprising:
(a) in-silico selecting a test agent that inhibits binding of an RNA binding form of IRP1 to an IRE but does not inhibit of IRP2 to an IRE; or
(b) in-silico selecting a test agent stabilizing a 4Fe-4S cluster form of IRP1.

11. The method of claim 10, further comprising providing said test agent and testing an anti-inflammatory activity of same.

12. The method of claim 11, wherein said testing is effected in-vitro.

13-16. (canceled)

17. The method of claim 1, wherein said agent comprises an RNA silencing agent.

18. The method of claim 1, wherein said agent is selected from the group consisting of a peptide and a small molecule.

19-37. (canceled)

38. The pharmaceutical composition of claim 3, wherein said agent comprises an RNA silencing agent.

39. The method of claim 10, wherein said agent is selected from the group consisting of a peptide and a small molecule.


SEQUENCE LISTING (12 sequences)

SEQ ID NO: 1 - DNA, 18 bp, Artificial Sequence
  Single strand DNA oligonucleotide
cttcctcacagagccagc

SEQ ID NO: 2 - DNA, 21 bp, Artificial Sequence
  Single strand DNA oligonucleotide
gatgcagacttcatcccaaga

SEQ ID NO: 3 - DNA, 20 bp, Artificial Sequence
  Single strand DNA oligonucleotide
agctcattcctccactcatg

SEQ ID NO: 4 - DNA, 20 bp, Artificial Sequence
  Single strand DNA oligonucleotide
acagacacagatgccagagg

SEQ ID NO: 5 - DNA, 20 bp, Artificial Sequence
  Single strand DNA oligonucleotide
gcatgcatccattgtctctg

SEQ ID NO: 6 - DNA, 24 bp, Artificial Sequence
  Single strand DNA oligonucleotide
acgtgtcctgtttgcccttgtatc

SEQ ID NO: 7 - DNA, 26 bp, Artificial Sequence
  Single strand DNA oligonucleotide
  misc_feature (26)..(26): n is a, c, g, or t
tctgtaaagagtggtccactgtgagn

SEQ ID NO: 8 - DNA, 24 bp, Artificial Sequence
  Single strand DNA oligonucleotide
cagcctctgttccacatacacttc

SEQ ID NO: 9 - DNA, 26 bp, Artificial Sequence
  Single strand DNA oligonucleotide
agaccctcacactcagatcatcttct

SEQ ID NO: 10 - DNA, 20 bp, Artificial Sequence
  Single strand DNA oligonucleotide
ctgctcctccacttggtggt

SEQ ID NO: 11 - DNA, 21 bp, Artificial Sequence
  Single strand DNA oligonucleotide
agccttccttcttgggtatgg

SEQ ID NO: 12 - DNA, 22 bp, Artificial Sequence
  Single strand DNA oligonucleotide
tcaacgtcacacttcatgatgg